(19) United States
(12) Patent Application Publication
(10) Pub. No.: US 2007/0161577 A1
(43) Pub. Date: Jul. 12, 2007

(54) Tethered dimers and trimers of 1,4-diphenylazetidin-2-ones

(76) Inventors: Eduardo J. Martinez, New York, NY (US); John J. Talley, Somerville, MA (US)

Correspondence Address: HESLIN ROTHENBERG FARLEY & MESITI PC, 5 COLUMBIA CIRCLE, ALBANY, NY, 12203, US

(21) Appl. No.: 10/569,561

(22) PCT Filed: Aug. 27, 2004
(86) PCT No.: PCT/US04/27813

§ 371 Date: Oct. 10, 2006

Related U.S. Application Data
(60) Provisional application No. 60/498,476, filed on Aug. 28, 2003.

(51) Int. Cl. A61K 31/7052; A61K 31/397; C07D 205/02; C07D 403/02

(52) U.S. Cl. 514/23; 514/210.02; 536/17.4; 540/200

(57) ABSTRACT

Derivatives of 1,4-diphenylazetidin-2-ones useful for the treatment of hypercholesterolemia are disclosed. The compounds are of the general formula

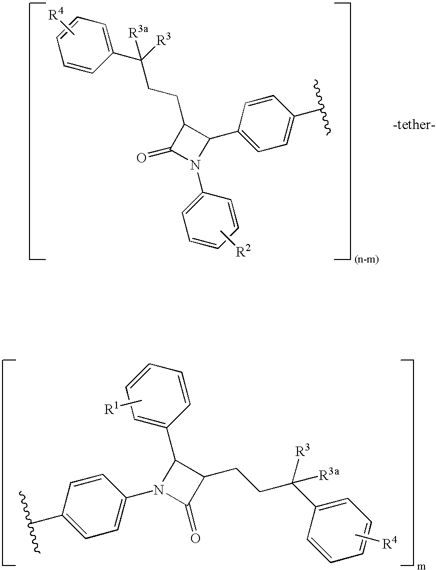

### FIELD OF THE INVENTION

The invention relates to a chemical genus of 1,4-diphenylazetidin-2-ones useful for the treatment of hypercholesterolemia.

### BACKGROUND OF THE INVENTION

1,4-Diphenylazetidin-2-ones and their utility for treating disorders of lipid metabolism are described in U.S. Pat. No. 6,498,156, USRE37721 and PCT application WO02/50027, the disclosures of which are incorporated herein by reference as they relate to utility. Perhaps the most well-known member of the class of 1,4-diphenylazetidin-2-one hypocholesterolemics is ezetimibe, which is sold as ZETIA™.

### SUMMARY OF THE INVENTION

In one aspect the invention relates to compounds of the general formula I: 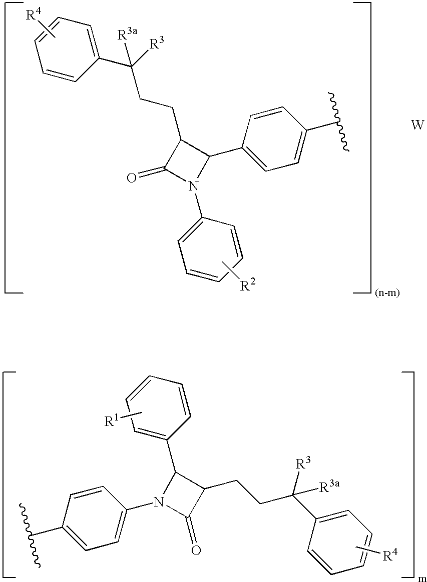
wherein
R¹ and R² are chosen from H, halogen, —OH, loweralkyl, —O-loweralkyl, —CN, —S-loweralkyl, amino, lower alkylamino, alkylsulfonyl, arylsulfonyl, acyl, a sugar, a glucuronide and a sugar carbamate;
R³ is chosen from H, —OH, fluoro and —O-loweralkyl;
R^{3a} is chosen from H and fluoro, or R^{3a} and R³ together are ═O;
R⁴ is chosen from H, halogen, —OH, loweralkyl, —O-loweralkyl, —CN, —S-loweralkyl, amino, lower alkylamino, alkylsulfonyl, arylsulfonyl and acyl; 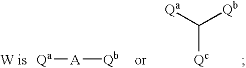
Q^{a}, Q^{b} and Q^{c} are independently chosen from a direct bond, —O—, —S—, —NH—, —CH₂O—, —CH₂NH—, —OCH₂CONH—, —OCH₂COO—, —C(═O)—, —CONH—, —NHCO—, —O(C═O)—, —(C═O)O—, —NHCONH—, —OCONH— and —NHCOO—;
n is 2or 3;
m is 0, 1, 2 or 3 and m=n; and
   A has a valency of n and is chosen from C₂ to C₂₀ hydrocarbon, substituted alkyl of 2 to 20 carbons, perfluoroalkyl of 2 to 20 carbons, substituted aryl, polyaryl of 3 to 20 aryl groups, substituted arylalkyl, oxaalkyl of four to fifty carbons, azaalkyl of four to fifty carbons, thiaalkyl of four to fifty carbons, a residue of an oligopeptide of two to twenty amino acids, a residue of a monosaccharide or of a polysaccharide of 2 to 100 saccharide residues; and, when Q^{a} and Q^{b} are —O(C═O)— or —NHCO—, A may additionally be methylene.

In a second aspect the invention relates to pharmaceutical formulations comprising a pharmaceutically acceptable carrier and a compound as above and, optionally, additionally comprising one or more of (1) an inhibitor of cholesterol biosynthesis; (2) a cholesterol ester transfer protein (CETP) inhibitor; (3) a bile acid sequestrant; (4) a nicotinic acid or derivative thereof; (5) a peroxisome proliferator-activated receptor activator; (6) an acylcoenzyme A:cholesterol acyltransferase (ACAT) inhibitor; and (7) an obesity control medication.

In a third aspect, the invention relates to methods for treating a disorder of lipid metabolism, including hyperlipidemia and arteriosclerotic symptoms; inhibiting the absorption of cholesterol from the intestine; reducing the blood plasma or serum concentrations of LDL cholesterol; reducing the concentrations of cholesterol and cholesterol ester in the blood plasma or serum; reducing blood plasma or serum concentrations of C-reactive protein (CRP), reducing blood plasma or serum concentrations of triglycerides; reducing blood plasma or serum concentrations of apolipoprotein B; increasing blood plasma or serum concentrations of high density lipoprotein (HDL) cholesterol; increasing the fecal excretion of cholesterol; treating a clinical condition for which a cholesterol absorption inhibitor is indicated and reducing the incidence of coronary heart disease-related events; reducing plasma or tissue concentration of at least one non-cholesterol sterol or 5α-stanol; treating or preventing vascular inflammation; preventing, treating, or ameliorating symptoms of Alzheimer's Disease; regulating the production or level of at least one amyloid β peptide in bloodstream and/or brain of a subject; regulating the amount of ApoE isoform 4 in the bloodstream and/or brain; preventing and/or treating obesity; and preventing or decreasing the incidence of xanthomas. The methods comprise administering a compound described above.

In a fourth aspect, the invention relates to methods and compositions for prevention or treatment of a cholesterol-associated tumor. The methods comprise administering a therapeutically effective amount of a compound of the invention to a patient at risk of developing a cholesterol-associated tumor or already exhibiting a cholesterol-associated tumor. The method also includes coadministering a therapeutically effective amount of a compound of the invention and at least one other anticancer agent.

In a fifth aspect, the invention relates to an article of manufacture comprising a container, instructions, and a pharmaceutical formulation as described above. The instructions are for the administration of the pharmaceutical formulation for a purpose chosen from: the prevention or treatment of a disorder of lipid metabolism; inhibiting the absorption of cholesterol from the intestine; reducing the plasma or tissue concentration of at least one non-cholesterol sterol or 5α-stanol; reducing the blood plasma or serum concentrations of LDL cholesterol; reducing the concentrations of cholesterol and cholesterol ester in the blood plasma or serum; increasing the fecal excretion of cholesterol; reducing the incidence of coronary heart disease-related events; reducing blood plasma or serum concentrations of C-reactive protein (CRP); treating or preventing vascular inflammation; reducing blood plasma or serum concentrations of triglycerides; increasing blood plasma or serum concentrations of HDL cholesterol; reducing blood plasma or serum concentrations of apolipoprotein B; preventing, treating, or ameliorating symptoms of Alzheimer's Disease; regulating the, production of amyloid β peptide; regulating the amount of ApoE isoform 4 in the bloodstream and/or brain; preventing and/or treating obesity; preventing or decreasing the incidence of xanthomas; and preventing or treating a cholesterol-associated tumor.

### DETAILED DESCRIPTION OF THE INVENTION

Compounds of the invention may be thought of as two or three 1,4-diphenylazetidin-2-one hypocholesterolemics “tethered” together. The tether is defined as W in the formula I above. The tether is divalent or trivalent and is comprised of a core “A”, and the points of attachment “Q”. “A” may be C₂ to C₂₀ hydrocarbon, substituted alkyl of 2 to 20 carbons, perfluoroalkyl of 2 to 20 carbons, substituted aryl, polyaryl of 3 to 20 aryl groups, substituted arylalkyl, oxaalkyl of four to fifty carbons, azaalkyl of four to fifty carbons, a residue of an oligopeptide of two to twenty amino acids or a residue of a monosaccharide or of a polysaccharide of 2 to 100 saccharide residues. Q^{a} and Q^{b} and, when present, Q^{c} may be independently a direct bond, —O—, —S—, —NH—, —CH₂O—, —CH₂NH—, —OCH₂CONH—, —OCH₂COO—, —C(═O)—, —CONH—, —NHCO—, —O(C═O)—, —(C═O)O—, —NHCONH—, —OCONH— or —NHCOO—. Additionally, when Q^{a} and Q^{b} are —O(C═O)— or —NHCO—, A may be methylene.

In the compounds of formula I, n may be 2 or 3; m may be 0-3. Of course, since the empirical formula I would not make sense unless the value of n minus m is zero or a positive number, m can only be 3 when n is 3, i.e. m=n. When m is zero, a subgenus of the following formula II, in which the tether attaches exclusively to the azetidine 4-phenyls, will result:

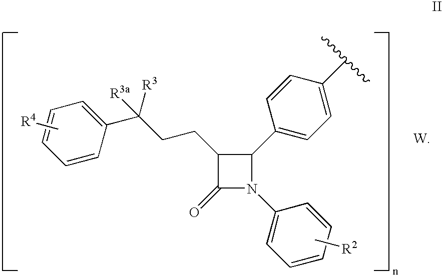

When m=n, a subgenus of the following formula III, in which the tether attaches exclusively to the azetidine 1-phenyls, will result:

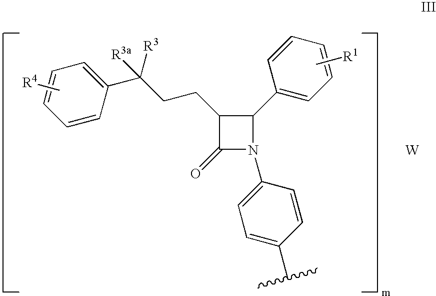

When m is not equal to n, and m is not zero, a subgenus will arise in which some tethers attach at 4-phenyl and some at 1-phenyl.

In an embodiment in which n is 3, and therefore W is trivalent, preferred compounds include those in which Q^{a}, Q^{b} and Q^{c} are independently chosen from —O—, —CH₂O—, —OCH₂COO—, —OCH₂CONH—, —(C═O)O—, and —NHCOO—; and A is a residue of a monosaccharide or of a polysaccharide of 2 to 100 saccharide residues, a branched oxaalkyl of four to fifty carbons or a monoazaalkyl of four to ten carbons and those in which Q^{a}, Q^{b} and Q^{c} are independently chosen from —CH₂O—, —CH₂NH—, —CONH—, —NHCO—, —OCH₂CONH—, —OCH₂COO—, —O(C═O)—, —(C═O)O—, —NHCONH—, —OCONH— and —NHCOO—; and A is an oligopeptide.

In an embodiment in which n is 2, and W is therefore divalent, preferred compounds are of formula:

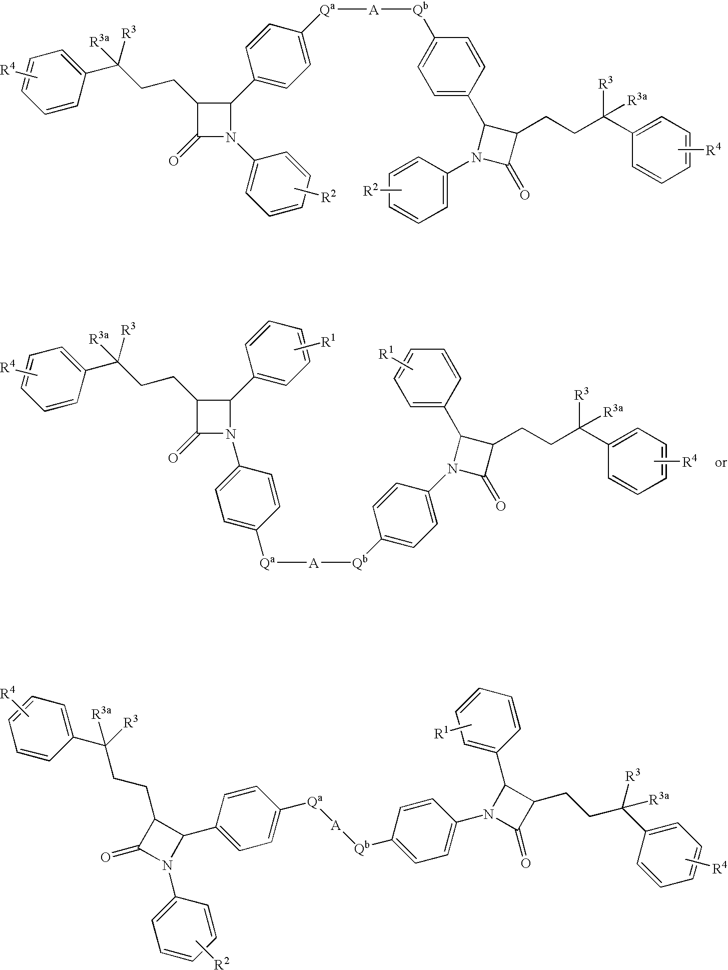

In these compounds Q^{a} and Q^{b} may be independently chosen from —O—, —CH₂O—, —OCH₂CONH—, —OCH₂COO—, —(C═O)O—, and —NHCOO—; and A is poly(oxyethylene) or a residue of a monosaccharide or of a polysaccharide of 2 to 100 saccharide residues; or Q^{a} and Q^{b} are independently chosen from —CH₂O—, —OCH₂CONH—, —OCH₂COO—, —CH₂NH—, —CONH—, —NHCO—, —O(C═O)—, —(C═O)O—, —NHCONH—, —OCONH— and —NHCOO—; and A is an oligopeptide.

Some examples may serve to illustrate the tethers: When Q^{a} and Q^{b} are both —NHCO— and A is (CH₂)₇, the tether W is divalent and the compound is an N,N′-disubstituted diamide of azelaic acid. When Q is —O(C═O)—, and A is methylene, the compounds are esters of malonic acid. When Q is —O— and A is 3-ethyl-3-azapentane, the tether W is trivalent and the compound is a triether of triethanolamine. When Q^{a} is —CONH—; Q^{b} is —NHCO— and A is 5-aminopentyl, the compounds are N^{α}-acylated lysineamides. When Q^{a} is —CONH—; Q^{b} is —NHCO— and A is a residue of an oligopeptide, the compounds are N^{α}-acylated peptide amides. Amino acids such as Ser, Thr, Lys, Orn, Arg, Glu, and Asp provide a scaffold for trivalent tethers. When A is a residue of a polysaccharide, either two or three hydroxyls of the saccharide may provide points of attachment.

In the azetidinone portion of the structures, substitutions include compounds wherein R¹ and R² are chosen from H, halogen, —OH, and methoxy; R³ is —OH; and R⁴ is fluoro and compounds wherein R¹ and R² are chosen from a sugar, a glucuronide and a sugar carbamate; R³ is —OH; and R⁴ is fluoro. A preferred azetidinone is:

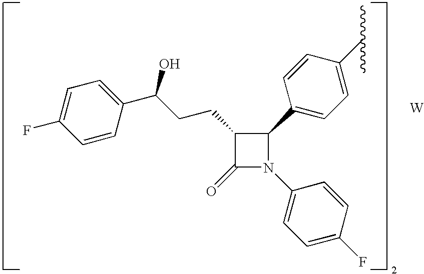

Compounds of the genus I above are inhibitors of cholesterol absorption from the intestine. As such they find utility in treating and preventing lipid disorders, such as hypercholesterolemia and hyperlipidemia. Because of their effect in lowering serum lipids, the compounds are useful in the treatment and prevention of atherosclerosis. Methods of the invention include the treatment of impaired lipid metabolism, hyperlipidemia, an arteriosclerotic symptom, and/or insulin resistance.

The compounds can be used advantageously in combination with other hypolipidemic agents, including inhibitors of cholesterol biosynthesis, such as the HMG-CoA reductase inhibitors. Preferred HMG-CoA reductase inhibitors would include the “statins”: lovastatin, simvastatin, mevastatin, atorvastatin, rosuvastatin, pravastatin, cerivastatin, pitavastatin and fluvastatin. A further listing of non-limiting examples of hypolipidemic agents that may be used in combination with the compounds of the present invention may be found in columns 5-6 of U.S. Pat. No. 6,498,156, the disclosure of which is incorporated herein by reference.

As described above, the formulation may additionally contain at least one bile acid sequestrant. Sequestrants include cholestyramine, colestipol and colesevelam hydrochloride. The formulation may also contain a nicotinic acid or derivative thereof. Nicotinic acid derivatives include niceritrol, nicofuranose and acipimox. The formulation may also contain a peroxisome proliferator-activated receptor activator, which may be a fibric acid derivative. Fibric acids include fenofibrate, clofibrate, gemfibrozil, ciprofibrate, bezafibrate, clinofibrate, binifibrate and lifibrol. The formulation may also contain a CETP inhibitor. Examples of such are the compounds identified as JTT-705 in Nature. 406 (6792):203-7 (2000 ) and CP-529,414 (torcetrapib), described in US20030186952 and WO2000017164. Examples of CETP inhibitors are also found in Current Opinion in Investigational Drugs. 4(3):291-297 (2003). The formulation may also contain an ACAT inhibitor. Examples of such are the compounds identified as avasimibe in Current Opinion in Investigational Drugs. 3(9):291-297 (2003), and CL-277,082 in Clin Pharmacol Ther. 48(2):189-94 (1990). The formulation may also contain an obesity control medication. Examples of obesity control medications include gut hormone fragment peptide YY₃₋₃₆ (PYY₃₋₃₆)(N. Engl. J Med. 349:941, 2003; IKPEAPGE DASPEELNRY YASLRHYLNL VTRQRY) or a variant thereof, glp-1 (glucagon-like peptide-1), exendin-4 (an inhibitor of glp-1), sibutramine, phentermine, phendimetrazine, benzphetamine hydrochloride (Didrex), orlistat (Xenical), diethylpropion hydrochloride (Tenuate), fluoxetine (Prozac), bupropion, ephedra, chromium, garcinia cambogia, benzocaine, bladderwrack (focus vesiculosus), chitosan, nomame herba, galega (Goat's Rue, French Lilac), conjugated linoleic acid, L-carnitine, fiber (psyllium, plantago, guar fiber), caffeine, dehydroepiandrosterone, germander (teucrium chamaedrys), B-hydroxy-β-methylbutyrate, ATL-962 (Alizyme PLC), and T71 (Tularik, Inc.; Boulder Colo.), a ghrelin antagonist, Acomplia (rimonabant), AOD9604, alpha-lipoic acid (alpha-LA), and pyruvate.

The present invention is also directed to methods of prevention or treatment of a cholesterol-associated tumor in patients who are either at risk of developing a cholesterol-associated tumor or already exhibit a cholesterol-associated tumor. The tumor may be either a benign or a malignant tumor of the prostate, breast, endometrium or colon. The compounds of the invention maybe co-administered with at least one other anticancer agent, which may be a steroidal antiandrogen, a non-steroidal antiandrogen, an estrogen, diethylstilbestrol, a conjugated estrogen, a selective estrogen receptor modulator (SERM), a taxane, or an LHRH analog. Tests showing the efficacy of the therapy and the rationale for combination therapy are presented in PCT application WO 2004/010948, the disclosure of which is incorporated herein by reference as it relates to utility.

The compounds of the invention may reduce both cholesterol levels in vivo and epoxycholesterol formation and thereby inhibit initiation and progression of benign and malignant cholesterol-associated tumors or cholesterol-associated cell growth or cell-masses. Compositions disclosed herein, for example, are useful for the treatment and/or prevention of benign prostatic hypertrophy, as well as tumors associated with prostate, colon, endometrial, or breast tissues.

Compositions of the invention comprise an effective dose or a pharmaceutically effective amount or a therapeutically effective amount of a compound described above and may additionally comprise at least one other anticancer agent, for the treatment or prevention of benign prostatic hypertrophy or other cholesterol-related benign or malignant tumors, particularly those associated with prostate, breast, endometrial or colon tissues. Examples of agents for use in compositions and methods of the invention include steroidal or non steroidal antiandrogens such as finasteride (PROSCAR®), cyproterone acetate (CPA), flutamide (4′-nitro-3′-trifluorormethyl isobutyranilide), bicalutamide (CASODEX®), and nilutamide; estrogens, diethylstilbestrol (DES); conjugated estrogens (e.g., PREMARIN®); selective estrogen receptor modulator (SERM) compounds such as tamoxifen, raloxifene, droloxifene, idoxifene; taxanes such as paclitaxel (TAXOL®) and docetaxel (TAXOTERE®); and LHRH analogs such as goserelin acetate (ZOLADEX®), and leuprolide acetate (LUPRON®).

Methods of the invention parallel the compositions and formulations. The methods comprise co-administering to a patient in need of treatment a therapeutically effective amount of an azetidinone according to the invention and one or more of: (a) a steroidal or non steroidal antiandrogen; (b) an estrogen; (c) diethylstilbestrol (DES); (d) a conjugated estrogen; (e) a selective estrogen receptor modulator (SERM); (f) a taxane; and (g) an LHRH analog. The term “selective estrogen receptor modulator” includes both estrogen agonist and estrogen antagonists and refers to compounds that bind with the estrogen receptor, inhibit bone turnover and prevent bone loss. In particular, estrogen agonists are compounds capable of binding to the estrogen receptor sites in mammalian tissue and mimicking the actions of estrogen in that tissue. Estrogen antagonists are compounds capable of binding to the estrogen receptor sites in mammalian tissue and blocking the actions of estrogen in that tissue. Exemplary SERMs are: tamoxifen (U.S. Pat. No. 4,536,516); 4-hydroxytamoxifen (U.S. Pat. No. 4,623,660); raloxifene (U.S. Pat. No. 4,418,068); idoxifene (U.S. Pat. No. 4,839,155; and droloxifene. For the taxanes see U.S. Pat. Nos. 6,395,770; 6,380,405; and 6,239,167. Azetidinones of the invention may also be combined with a steroidal or non steroidal antiandrogen, as described above.

Compounds of the invention have the advantage that they cholesterol and/or LDL levels but the compounds themselves are not appreciably absorbed into the mammalian circulation upon oral administration. As a result of the low-to-insignificant serum levels, fewer side-effects, such as drug-drug interactions, are observed.

The compounds of the invention are neutral, acidic or basic, depending on the functionality found in W. When acidic or basic, they may be presented as salts, and the term “pharmaceutically acceptable salt” refers to salts derived from non-toxic acids and bases. Suitable pharmaceutically acceptable acid-derived anions for the basic compounds of the present invention include hydroxide, acetate, benzenesulfonate (besylate), benzoate, bicarbonate, bisulfate, carbonate, camphorsulfonate, citrate, ethanesulfonate, fumarate, gluconate, glutamate, glycolate, bromide, chloride, isethionate, lactate, maleate, malate, mandelate, methanesulfonate, mucate, nitrate, pamoate, pantothenate, phosphate, succinate, sulfate, taitrate, trifluoroacetate, p-toluenesulfonate, acetamidobenzoate, adipate, alginate, aminosalicylate, anhydromethylenecitrate, ascorbate, aspartate, calcium edetate, camphorate, camsylate, caprate, caproate, caprylate, cinnamate, cyclamate, dichloroacetate, edetate (EDTA), edisylate, embonate, estolate, esylate, fluoride, formate, gentisate, gluceptate, glucuronate, glycerophosphate, glycolate, glycollylarsanilate, hexylresorcinate, hippurate, hydroxynaphthoate, iodide, lactobionate, malonate, mesylate, napadisylate, napsylate, nicotinate, oleate, orotate, oxalate, oxoglutarate, palmitate, pectinate, pectinate polymer, phenylethylbarbiturate, picrate, pidolate, propionate, rhodanide, salicylate, sebacate, stearate, tannate, theoclate, tosylate, and the like. When the compounds contain an acidic side chain, suitable pharmaceutically acceptable base addition salts for the compounds of the present invention include metallic salts made from aluminum, calcium, lithium, magnesium, potassium, sodium and zinc or organic salts made from lysine, N,N′-dibenzylethylenediamine, chloroprocaine, choline, diethanolanmine, ethylenediamine, meglumine (N-methylglucamine) and procaine. Other base addition salts includes those made from: arecoline, arginine, barium, benethamine, benzathine, betaine, bismuth, clemizole, copper, deanol, diethylamine, diethylaminoethanol, epolamine, ethylenediamine, ferric, ferrous, glucamine, glucosamine, histidine, hydrabamine, imidazole, isopropylamine, manganic, manganous, methylglucamine, morpholine, morpholineethanol, n-ethylmorpholine, n-ethylpiperidine, piperazine, piperidine, polyamine resins, purines, theobromine, triethylamine, trimethylamine, tripropylamine, trolamine, and tromethamine.

The desired salt may be obtained by methods well known to persons of skill. Although pharmaceutically acceptable counter ions will be preferred for preparing pharmaceutical formulations, other anions are quite acceptable as synthetic intermediates. Thus salts may be made from pharmaceutically undesirable anions, such as iodide, oxalate, trifluoromethanesulfonate, heavy metals and the like, when such salts are chemical intermediates.

Definitions

Throughout this specification the terms and substituents retain their definitions.

Alkyl is intended to include linear, branched, or cyclic hydrocarbon structures and combinations thereof. Lower alkyl refers to alkyl groups of from 1 to 6 carbon atoms. Examples of lower alkyl groups include methyl, ethyl, propyl, isopropyl, butyl, s- and t-butyl and the like. Preferred aikyl groups are those of C₂₀ or below. Cycloalkyl is a subset of alkyl and includes cyclic hydrocarbon groups of from 3 to 8 carbon atoms. Examples of cycloalkyl groups include c-propyl, c-butyl, c-pentyl, norbornyl, adamantyl and the like.

C₁ to C₂₀ Hydrocarbon includes alkyl, cycloalkyl, alkenyl, alkynyl, aryl and combinations thereof. Examples include phenethyl, cyclohexylmethyl, camphoryl and naphthylethyl.

Alkoxy or alkoxyl refers to groups of from 1 to 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. Lower-alkoxy refers to groups containing one to four carbons.

Oxaalkyl refers to alkyl residues in which one or more carbons (and their associated hydrogens) have been replaced by oxygen. Examples include methoxypropoxy, 3,6,9-trioxadecyl and the like. The term oxaalkyl is intended as it is understood in the art [see Naming and Indexing of Chemical Substances for Chemical Abstracts, published by the American Chemical Society, ¶196, but without the restriction of ¶127(a)], i.e. it refers to compounds in which the oxygen is bonded via a single bond to its adjacent atoms (forming ether bonds). Similarly, thiaalkyl and azaalkyl refer to alkyl residues in which one or more carbons have been replaced by sulfur or nitrogen, respectively. Examples include ethylaminoethyl and methylthiopropyl.

Acyl refers to groups of from 1 to 8 carbon atoms of a straight, branched, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like. Lower-acyl refers to groups containing one to four carbons.

Aryl and heteroaryl mean a 5- or 6-membered aromatic or heteroaromatic ring containing 0-3 heteroatoms selected from O, N, or S; a bicyclic 9- or 10-membered aromatic or heteroaromatic ring system containing 0-3 heteroatoms selected from O, N, or S; or a tricyclic 13- or 14-membered aromatic or heteroaromatic ring system containing 0-3 heteroatoms selected from O, N, or S. Aromatic 6- to 14-membered carbocyclic rings include, e.g., benzene, naphthalene, indane, tetralin, and fluorene and the 5- to 10-membered aromatic heterocyclic rings include, e.g., imidazole, pyridine, indole, thiophene, benzopyranone, thiazole, fuiran, benzimidazole, quinoline, isoquinoline, quinoxaline, pyrimidine, pyrazine, tetrazole and pyrazole. Polyaryls, as the name implies, are compounds comprising a plurality of aryl residues and no aliphatic residues. The most common lower polyaryl is biphenyl. Polyaryls of 3 to 20 would include terphenyl and the like.

Arylalkyl means an alkyl residue attached to an aryl ring. Examples are benzyl, phenethyl and the like.

Substituted alkyl, aryl, cycloalkyl, heterocyclyl etc. refer to alkyl, aryl, cycloalkyl, or heterocyclyl wherein up to three H atoms in each residue are replaced with halogen, haloalkyl, hydroxy, loweralkoxy, carboxy, carboalkoxy (also referred to as alkoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, phenyl, benzyl, heteroaryl, phenoxy, benzyloxy, or heteroaryloxy.

The term “halogen” means fluorine, chlorine, bromine or iodine.

The term “sugar” is used in its normal sense, as defined in Hawley's Condensed Chemical Dictionary, 12^{th} Edition, Richard J. Lewis, Sr.; Van Nostrand Reinhold Co. New York. It encompasses any carbohydrate comprised of one or two saccharose groups. The monosaccharide sugars (often called simple sugars) are composed of chains of 2-7 carbon atoms. One of the carbons carries aldehydic or kcetonic oxygen, which may be combined in acetal or ketal forms. The remaining carbons usually have hydrogen atoms and hydroxyl groups. Among monosaccharides which would be considered within the term “sugars” as intended in this application, are arabinose, ribose, xylose, ribulose, xylulose, deoxyribose, galactose, glucose, mannose, fructose, sorbose, tagatose, fucose, quinovose, rhamnose, manno-heptulose and sedoheptulose. Among the disaccharides are sucrose, lactose, maltose, and cellobiose. Unless specifically restricted, the general term “sugar” refers to both D-sugars and L-sugars.

The term “polysaccharide” refers to condensation polymers in which monosaccharides or their derivatives, such as aminosugars, uronic acids and sulfonic acid derivatives, have been glycosidically linked with the elimination of water. Polysaccharides include polymers of glucosamine, N-acetyl-D-glucosamine, hyaluronic acid and chondroitin sulfate, as well as polymers of the common sugars.

The term “glucuronide” is also used in its normal sense to refer to a glycoside of glucuronic acid.

The term “sugar carbamate” refers to mono-, di- and oligosaccharides in which one or more hydroxyls have been derivatized as carbamates, particularly as phenyl carbamates and substituted phenyl carbamates. [See Detmers et al. Biochim Biolphys. Acta 1486, 243-252 (2000), which is incorporated herein by reference.] A preferred sugar carbamate is:

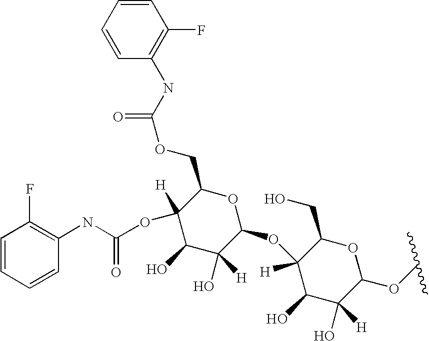

The term “a residue of a polysaccharide” when used to describe “A”, refers to a polysaccharide (as defined above) minus the functional groups that are considered part of “Q”. For example, in the molecule illustrated below: 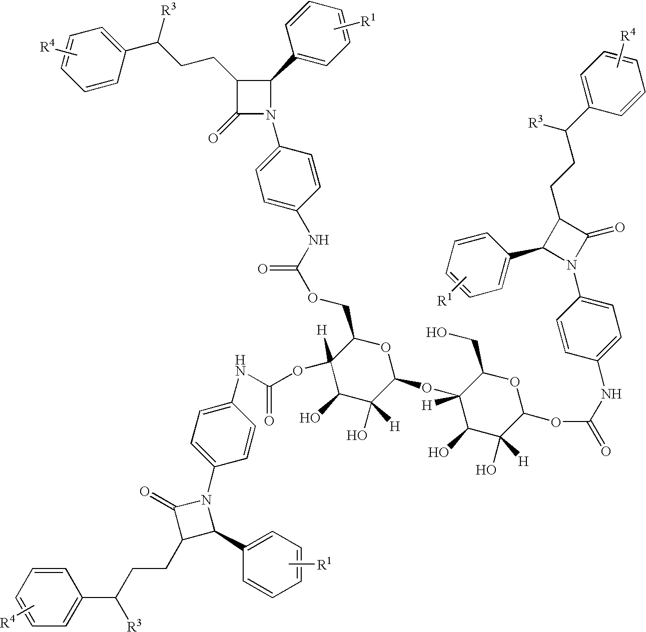
after one subtracts the three carbamate linkages —OCONH— that constitute “Q”, the structure of A that remains is: 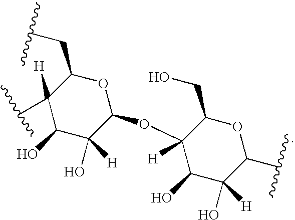
This is not sensu stricto a polysacchararide, since it lacks three hydroxyls to be cellobiose. This and similar structures of polysaccharides that lack two or three hydroxyls at the points of attachment of “Q” to “A” are referred to herein as “residues of polysaccharides”.

Similarly, the term “a residue of an oligopeptide” refers to an oligopeptide (as defined below) minus the finctional groups that are considered part of “Q”. For example, in the molecule illustrated below: 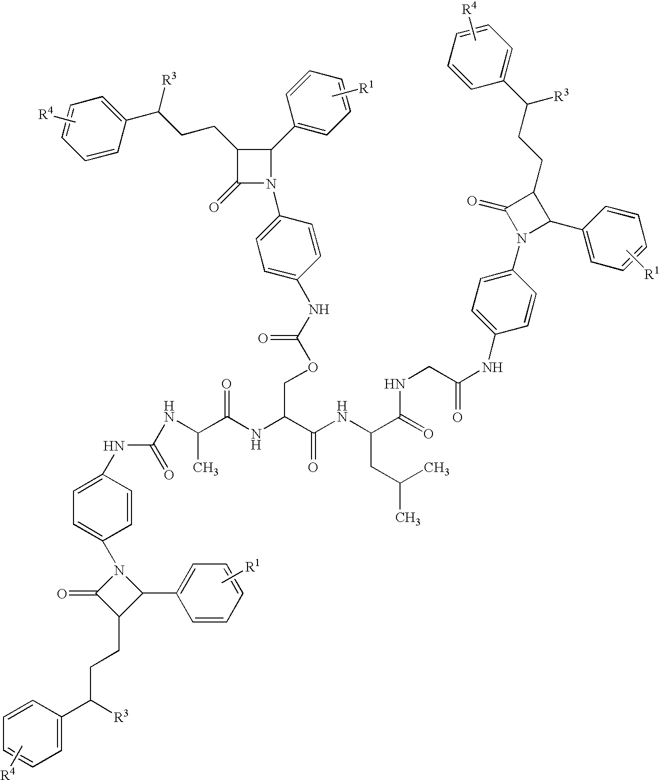
after one subtracts the urea —NHCONH— that is Q^{a}, the carbamate —OCONH— that is Q^{b} and the amide —CONH— that is Q^{c}, the structure of A that remains is:

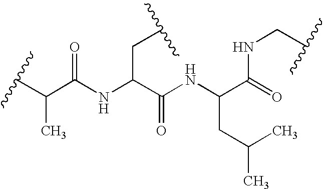

This is not sensu stricto an oligopeptide, since it lacks the N-terminal amine, the carboxy-terminal COOH and the side chain hydroxyl of Ser to be the tetrapeptide HAlaSerLeuGlyOH. This and similar structures of oligopeptides that lack two or three finctional groups at the points of attachment of “Q” to “A” are referred to herein as “residues of oligopeptides”. One might also refer to them as oligopeptide fragments.

Oligopeptide refers to oligomers of amino acids, and is intended to include the racemates and all optical isomers of the constituent amino acids. The amino acids include alanine, asparagine, aspartic acid, arginine, cysteine, phenylglycine, glutamic acid, glutamine, glycine, histidine, isoleucine, leucine, lysine, methionine, phenylalanine, proline, serine, threonine, tryptophan, tyrosine, valine, and sarcosine. The amino acids also include ω-amino acids of three to ten carbons, such as β-alanine, γ-aminobutyric acid and ε-amino caproic acid. Individual amino acids within the peptide may be protected, for example aspartic acid-β-t-butyl ester, N^{g}-Mtr-arginine, S -trityl-cysteine, glutamic acid-γ-t-butyl ester, N-γ-trityl-glutamine, N^{im}-trityl-histidine, N^{ε}-Boc-lysine, O-t-butyl-serine and Nⁱⁿ-Boc-tryptophan.

The term “prodrug” refers to a compound that is made more active in vivo. Since the compounds of the invention are minimally absorbed into the systemic circulation, activation in vivo may come about by chemical action or through the intermediacy of enzymes and microflora in the GI tract.

It will be recognized that the compounds of this invention can exist in radiolabeled form, i.e., the compounds may contain one or more atoms containing an atomic mass or mass number different from the atomic mass or mass number usually found in nature. Radioisotopes of hydrogen, carbon, phosphorous, fluorine, and chlorine include ³H, ¹⁴C, ³⁵S, ¹⁸F, and ³⁶Cl, respectively. Compounds that contain those radioisotopes and/or other radioisotopes of other atoms are within the scope of this invention. Tritiated, i.e. ³H, and carbon-14, i.e., ¹⁴C, radioisotopes are particularly preferred for their ease in preparation and detectability. Radiolabeled compounds of Formulas I-VII of this invention and prodrugs thereof can generally be prepared by methods well known to those skilled in the art. Conveniently, such radiolabeled compounds can be prepared by carrying out the procedures disclosed in the Examples and Schemes by substituting a readily available radiolabeled reagent for a non-radiolabeled reagent.

The terms “methods of treating or preventing” mean amelioration, prevention or relief from the symptoms and/or effects associated with lipid disorders. The term “preventing” as used herein refers to administering a medicament beforehand to forestall or obtund an acute episode. The person of ordinary skill in the medical art (to which the present method claims are directed) recognizes that the term “prevent” is not an absolute term. In the medical art it is understood to refer to the prophylactic administration of a drug to substantially diminish the likelihood or seriousness of a condition, and this is the sense intended in applicants' claims. As used herein, reference to “treatment” of a patient is intended to include prophylaxis. Throughout this application, various references are referred to within parentheses or square brackets. The disclosures of these publications in their entireties are hereby incorporated by reference as if written herein.

The term “mammal” is used in its dictionary sense. The term “mammal” includes, for example, mice, hamsters, rats, cows, sheep, pigs, goats, and horses, monkeys, dogs (e.g., Canis familiaris), cats, rabbits, guinea pigs, and primates, including humans.

The compounds may be use to treat or prevent vascular inflammation, as described in US published application 20030119757; to prevent, treat, or ameliorate symptoms of Alzheiner's Disease and to regulate the production or level of amyloid β peptide and ApoE isoform 4, as described in U.S. Pat. No. 6,080,778 and US published application 20030013699; and to prevent or decrease the incidence of xanthomas, as described in US published application 20030119809. The disclosures of all are incorporated herein by reference.

The compounds described herein contain two or more asymmetric centers and may thus give rise to enantiomers, diastereomers, and other stereoisomeric forms. Each chiral center may be defmed, in terms of absolute stereochemistry, as (R)- or (S)-. The present invention is meant to include all such possible isomers, as well as, their racemic and optically pure forms. Optically active (R)- and (S)-, or (D)- and (L)- isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

The graphic representations of racemic, ambiscalemic and scalemic or enantiomerically pure compounds used herein are taken from Maehr J. Chem. Ed. 62, 114-120 (1985): solid and broken wedges are used to denote the absolute configuration of a chiral element; wavy lines and single thin lines indicate disavowal of any stereochemical implication which the bond it represents could generate; solid and broken bold lines are geometric descriptors indicating the relative configuration shown but denoting racemic character; and wedge outlines and dotted or broken lines denote enantiomerically pure compounds of indeterminate absolute configuration. Thus, the formula X is intended to encompass both of the pure enantiomers of that pair:

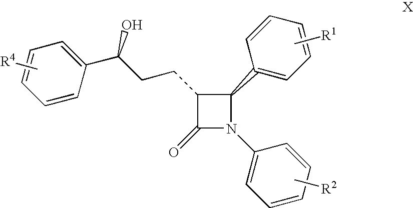

Means either pure R,S,R: 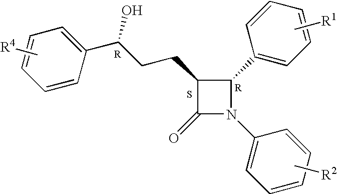
or pure S,R,S: 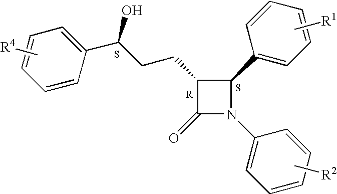
whereas 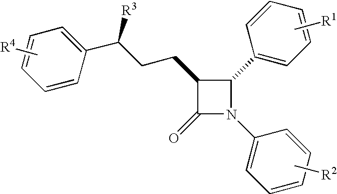
refers to a racemic mixture of S,R,S and S,S,R, i.e. having a trans relative configuration on the beta lactam ring.

The term “enantiomeric excess” is well known in the art and is defined for a

$${ee}_{a} = ( \frac{conc . of a - conc . of b}{conc . of a + conc . of b} ) \times 100$$

resolution of ab ? a+b as

The term “enantiomeric excess” is related to the older term “optical purity” in that both are measures of the same phenomenon. The value of ee will be a number from 0 to 100, zero being racemic and 100 being pure, single enantiomer. A compound which in the past might have been called 98% optically pure is now more precisely described as 96% ee; in other words, a 90% ee reflects the presence of 95% of one enantiomer and 5% of the other in the material in question.

The configuration of any carbon-carbon double bond appearing herein is selected for convenience only and is not intended to designate a particular configuration; thus a carbon-carbon double bond depicted arbitrarily herein as trans may be cis, trans, or a mixture of the two in any proportion.

Terminology related to “protecting”, “deprotecting” and “protected” functionalities occurs throughout this application. Such terminology is well understood by persons of skill in the art and is used in the context of processes which involve sequential treatment with a series of reagents. In that context, a protecting group refers to a group which is used to mask a functionality during a process step in which it would otherwise react, but in which reaction is undesirable. The protecting group prevents reaction at that step, but may be subsequently removed to expose the original functionality. The removal or “deprotection” occurs after the completion of the reaction or reactions in which the fumctionality would interfere. Thus, when a sequence of reagents is specified, as it is in the processes of the invention, the person of ordinary skill can readily envision those groups that would be suitable as “protecting groups”. Suitable groups for that purpose are discussed in standard textbooks in the field of chemistry, such as Protective Groups in Organic Synthesis by T. W. Greene [John Wiley & Sons, New York, 1991], which is incorporated herein by reference. Particular attention is drawn to the chapters entitled “Protection for the Hydroxyl Group, Including 1,2- and 1,3-Diols” (pages 10-86).

The abbreviations Me, Et, Ph, Tf, Ts and Ms represent methyl, ethyl, phenyl, trifluoromethanesulfonyl, toluensulfonyl and methanesulfonyl respectively. A comprehensive list of abbreviations utilized by organic chemists (i.e. persons of ordinary skill in the art) appears in the first issue of each volume of the Journal of Organic Chemistry. The list, which is typically presented in a table entitled “Standard List of Abbreviations” is incorporated herein by reference.

While it may be possible for the compounds of formula (I) to be administered as the raw chemical, it is preferable to present them as a pharmaceutical composition. According to a further aspect, the present invention provides a pharmaceutical composition comprising a compound of formula (I) or a pharmaceutically acceptable salt or solvate thereof, together with one or more pharmaceutically carriers thereof and optionally one or more other therapeutic ingredients. The carrier(s) must be “acceptable” in the sense of being compatible with the other ingredients of the formulation and not deleterious to the recipient thereof.

The formulations include those suitable for oral, parenteral (including subcutaneous, intradermal, intramuscular, intravenous and intraarticular), rectal and topical (including dermal, buccal, sublingual and intraocular) administration. The most suitable route may depend upon the condition and disorder of the recipient. The formulations may conveniently be presented in unit dosage form and may be prepared by any of the methods well known in the art of pharmacy. All methods include the step of bringing into association a compound of formula (I) or a pharmaceutically acceptable salt or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

The pharmaceutical compositions may include a “pharmaceutically acceptable inert carrier”, and this expression is intended to include one or more inert excipients, which include starches, polyols, granulating agents, microcrystalline cellulose, diluents, lubricants, binders, disintegrating agents, and the like. If desired, tablet dosages of the disclosed compositions may be coated by standard aqueous or nonaqueous techniques, “Pharmaceutically acceptable carrier” also encompasses controlled release means.

Compositions of the present invention may also optionally include other therapeutic ingredients, anti-caking agents, preservatives, sweetening agents, colorants, flavors, desiccants, plasticizers, dyes, and the like. Any such optional ingredient must, of course, be compatible with the compound of the invention to insure the stability of the formulation.

Examples of excipients for use as the pharmaceutically acceptable carriers and the pharmaceutically acceptable inert carriers and the aforementioned additional ingredients include, but are not limited to:

BINDERS: corn starch, potato starch, other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch (e.g., STARCH 1500® and STARCH 1500 LM®, sold by Colorcon, Ltd.), hydroxypropyl methyl cellulose, microcrystalline cellulose (e.g. AVICEL™, such as, AVICEL-PH-101™, -103™ and -105™, sold by FMC Corporation, Marcus Hook, Pa., USA), or mixtures thereof;

FILLERS: talc, calcium carbonate (e.g., granules or powder), dibasic calcium phosphate, tribasic calcium phosphate, calcium sulfate (e.g., granules or powder), microcrystalline cellulose, powdered cellulose, dextrates, kaolin, mannitol, silicic acid, sorbitol, starch, pre-gelatinized starch, or mixtures thereof;

DISINTEGRANTS: agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, or mixtures thereof;

LUBRICANTS: calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W.R. Grace Co., Baltimore, Md. USA), a coagulated aerosol of synthetic silica (Deaussa Co., Plano, Tex. USA), a pyrogenic silicon dioxide (CAB-O-SIL, Cabot Co., Boston, Mass. USA), or mixtures thereof;

ANTI-CAKING AGENTS: calcium silicate, magnesium silicate, silicon dioxide, colloidal silicon dioxide, talc, or mixtures thereof;

ANTIMICROBIAL AGENTS: benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenylmercuric acetate, phenylmercuric nitrate, potassium sorbate, propylparaben, sodium benzoate, sodium dehydroacetate, sodium propionate, sorbic acid, thimersol, thymo, or mixtures thereof; and

COATING AGENTS: sodium carboxymethyl cellulose, cellulose acetate phthalate, ethylcellulose, gelatin, pharmaceutical glaze, hydroxypropyl cellulose, hydroxypropyl methylcellulose, hydroxypropyl methyl cellulose phthalate, methylcellulose, polyethylene glycol, polyvinyl acetate phthalate, shellac, sucrose, titanium dioxide, carnuba wax, microcrystalline wax, or mixtures thereof

The dose range for adult humans is generally from 0.005 mg to 10 g/day orally. Tablets or other forms of presentation provided in discrete units may conveniently contain an amount of compound of the invention which is effective at such dosage or as a multiple of the same, for instance, units containing 5 mg to 500 mg, usually around 10 mg to 200 mg. The precise amount of compound administered to a patient will be the responsibility of the attendant physician. However, the dose employed will depend on a number of factors, including the age and sex of the patient, the precise disorder being treated, and its severity.

Combination therapy can be achieved by administering two or more agents, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present in within the patient's body at the same time, this need not be so. Combination therapy can also include two or more administrations of one or more of the agents used in the combination. For example, if agent X and agent Y are used in a combination, one could administer them sequentially in any combination one or more times, e.g., in the order X-Y-X, X-X-Y, Y-X-Y, Y-Y-X, X-X-Y-Y, etc.

A pharmaceutical composition according to the invention may also comprise, as a frrther active compound, one or more antidiabetics, hypoglycemically active compounds, HMGCoA reductase inhibitors, cholesterol absorption inhibitors, PPAR gamma agonists, PPAR alpha agonists, PPAR alpha/gamma agonists, fibrates, MTP inhibitors, bile acid absorption inhibitors, CETP inhibitors, polymeric bile acid adsorbers, LDL receptor inducers, ACAT inhibitors, antioxidants, lipoprotein lipase inhibitors, ATP citrate lyases inhibitors, squalene synthetase inhibitors, lipoprotein(a) antagonists, lipase inhibitors, insulins, sulphonyl ureas, biguanides, meglitinides, thiolidindiones, a-glucosidase inhibitors, active compounds which act on the ATP-dependent potassium channel of the beta cells, CART agonists, NPY agonists, MC4 agonists, orexin agonists, H3 agonists, TNF agonists, CRF agonists, CRF BP agonists, urocortin agonists, MSH (melanocyte-stimulating hormone) agonists, CCK agonists, serotonin-reuptake inhibitors, mixed serotonin and noradrenergic compounds, 5HT agonists, bombesin agonists, galanin agonists, growth hormones, growth hormone-releasing compounds, TRH agonists, decoupling protein 2- or 3-modulators, leptin agonists, DA agonists, lipase/amylase inhibitors, PPAR modulators, R×R modulators or TR-β-agonists or amphetamines.

In Vivo Assay of Hypolipidemic Agents using the Rat Cholesterol Absorption Model. This model is based on models described by Burnett et al (2002), Bioorg Med Chem Lett. 2002 Feb. 11; 12(3):315-8 and J Lipid Res. 1999 October;40(10):1747-57. Female Sprague-Dawley rats weighing 150-250 g are separated into groups of 3 and fasted overnight. The animals (4-6/group) are dosed perorally with 300 μL test compounds in olive oil or suitable vehicle. Thirty minutes later, 3-5 microCuries ³H-cholesterol per rat are delivered perorally in 300 μL olive oil . After three hours, 200 μL serum is collected, vortexed with scintillation fluid, and measured for radioactivity in a scintillation counter. Percent inhibition is defmed as 100*(1-Cₜₑₛₜ/C_{ctrl}), where Cₜₑₛₜ and C_{ctrl} refer to ³H levels in serum for the test compound and for the vehicle only control, respectively. Percent inhibition values are reported for a fixed dose. The ED₅₀ is the dose at which the half-maximnal effect on serum ³H levels is observed for a given test compound.

In Vivo Assay of Hypolipidemic Agents using the Mouse Cholesterol Absorption Model. Female CD-1 mice weighing 20-30 g are separated into groups of 3-8 and fasted overnight. The animals (3-8/group) are dosed perorally with 200 μL test compound in olive oil or suitable vehicle. Thirty minutes later, 3-5 microCuries ³H-cholesterol per mouse are delivered perorally in 200 μL olive oil. After three hours, 100 μL serum is collected, vortexed with scintillation fluid, and measured for radioactivity in a scintillation counter. Percent inhibition and ED₅₀ are defined as in the Rat Cholesterol Absorption Model above.

In Vivo Assay of Hypolipidemic Agents Using Se Hyperlipidemic Hamster: Hamsters are separated into groups of six and given a controlled cholesterol diet (Purina Chow #5001 containing 0.5% cholesterol) for seven days. Diet consumption is monitored to determine dietary cholesterol exposure in the face of test compounds. The animals are dosed with the test compound once daily beginning with the initiation of diet. Dosing is by oral gavage of 0.2 mL of corn oil alone (control group) or solution (or suspension) of test compound in corn oil. All animals moribund or in poor physical condition are euthanized. After seven days, the animals are anesthetized by intramuscular (IM injection of ketamine and sacrificed by decapitation. Blood is collected into vacutainer tubes containing EDTA for plasma lipid analysis and the liver excised for tissue lipid analysis. Lipid analysis is conducted as per published procedures [Schnitzer-Polokoff, R., et al, Comp. Biochem. Physiol., 99A, 4, 665-670 (1991)] and data are reported as percent reduction of lipid versus control.

In Vivo Assay of Hypolipidemic Agents using the Hamster Acute Cholesterol Absorption Model. Male Syrian Hamsters weighing 120 g are separated into groups of 3-6 and fasted overnight. The animals (3-6/group) are dosed perorally with 200 μL test compound in olive oil or suitable vehicle. Thirty minutes later, 3-5 microCuries ³H-cholesterol per hamster are delivered perorally in 200 μL olive oil. After three hours, 100-200 μL serum is collected, vortexed with scintillation fluid, and measured for radioactivity in a scintillation counter. Percent inhibition and ED₅₀ are defined as in the Rat Cholesterol Absorption Model above.

The bioabsorption of the compounds herein described may be examined using the Caco-2 cell monolayer model of Hilger et al. [Pharm. Res. 7, 902 (1990)]. To study the pharmacokinetics of compounds, bioavailability studies are carried out in rats. Compounds are prepared in suitable formulations: 5% ethanol in olive oil for oral administration and 2% DMSO: 20% cyclodextrins in H₂O for intravenous administration. Compounds are administered intravenously via tail vein injection and orally by gavage to independent groups of CD rats (200-250 g). Serum is collected at various time points and assayed for the presence of compounds using an LC/MS/MS detection method. Samples are diluted 15-fold in 30% acetonitrile in water, then injected (35 μL) into a 3.2 ml/min flow of 5% methanol in water onto a sample extraction cartridge (Waters Oasis HLB Direct Connect), washed for 30 seconds, then loaded onto a reverse phase HPLC column (Thermo Electron Betasil C18 Pioneer 50×2.1 mm, 5 um particle size). Samples are eluted from the reverse phase HPLC column with a gradient: (Mobile Phase A: 5 mM ammonium acetate in dH₂O, Mobile Phase B: 20% methanol in acetonitrile; 40% B ramping to 95% B over 4 minutes, and holding for 3 minutes, then returning to initial conditions to re-equilibrate the column for 1 min, all at a flow rate of 0.3 ml/min.). A Micromass Quattro Micro (Waters Corp.; Milford, Mass.) triple quadrupole mass spectrometer operated in MRM mode is used for detection. Concentrations are calculated based on standard concentration curves of compounds. MassLynx software (Waters, Corp.; Milford, Mass.) is used to calculate the absolute concentration of test compound in each serum sample. A concentration versus time plot is generated from the data in Microsoft Excel, Summuit Software PK Solutions 2.0 or GraphPad Prism (GraphPad Software, Inc., San Diego, Calif.) to generate pharmacokinetic curves. An area under the curve (AUCₙ, n=length of experiment in minutes or hours) is calculated from the concentration vs. time data by the software using the trapezoid method for both the orally and intravenously dosed animals. Oral Bioavailability (F) over the length of the experiment is calculated using the equation:
F=(AUCₒᵣₐₗ*Dose_{i.v.})/(AUC_{i.v.}*Doseₒᵣₐₗ)

In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants that are in themselves known, but are not mentioned here. The starting materials, in the case of suitably substituted azetidinones, may be obtained by the methods described in WO 02/50027, WO 97/16424, WO 95/26334, WO 95/08532 and WO 93/02048, the disclosures of which are incorporated herein by reference.

Processes for obtaining exemplary compounds of the invention are presented below.

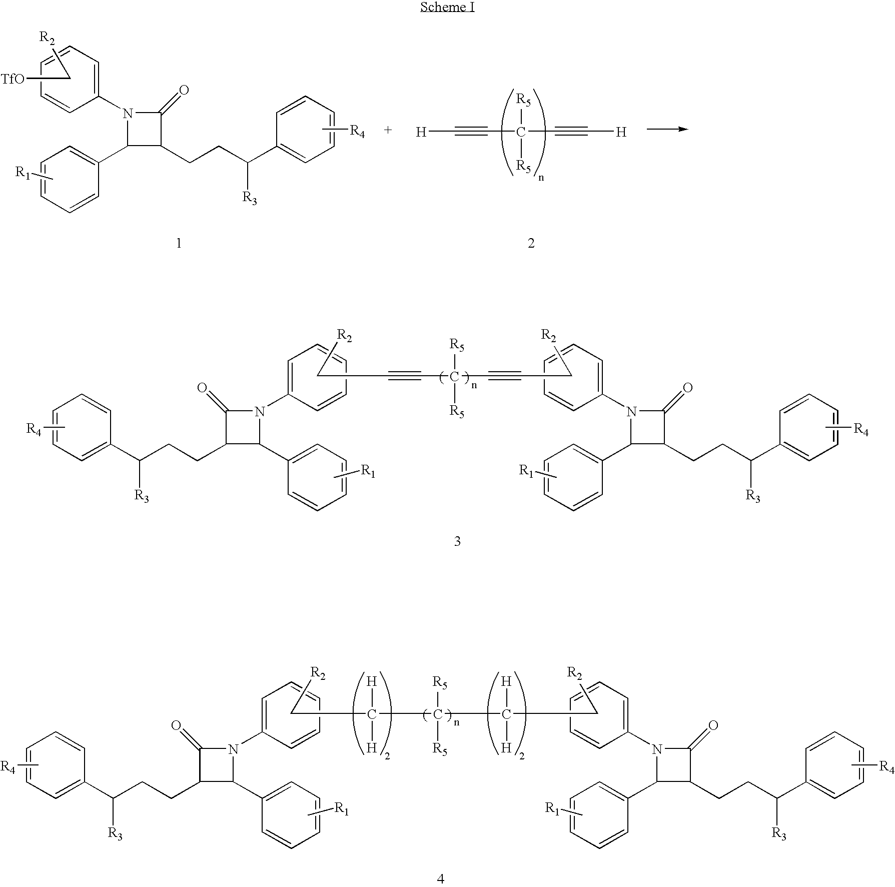

Illustrated in Scheme I is the general method for the preparation of cholesterol absorption inhibitors of the general formula 3 and 4. The procedure commences with the Sonogashira coupling of triflate 1 (derived from the corresponding phenol by treatment with N-phenyltrifluoromethanesulfonimide) with bis-acetylenes of the type 2 to afford the dimeric cholesterol absorption inhibitors of formula 3. Hydrogenation of the triple bonds of 3 in the presence of a suitable catalyst, such as palladium on carbon, affords the saturated cholesterol absorption inhibitors of formula 4.

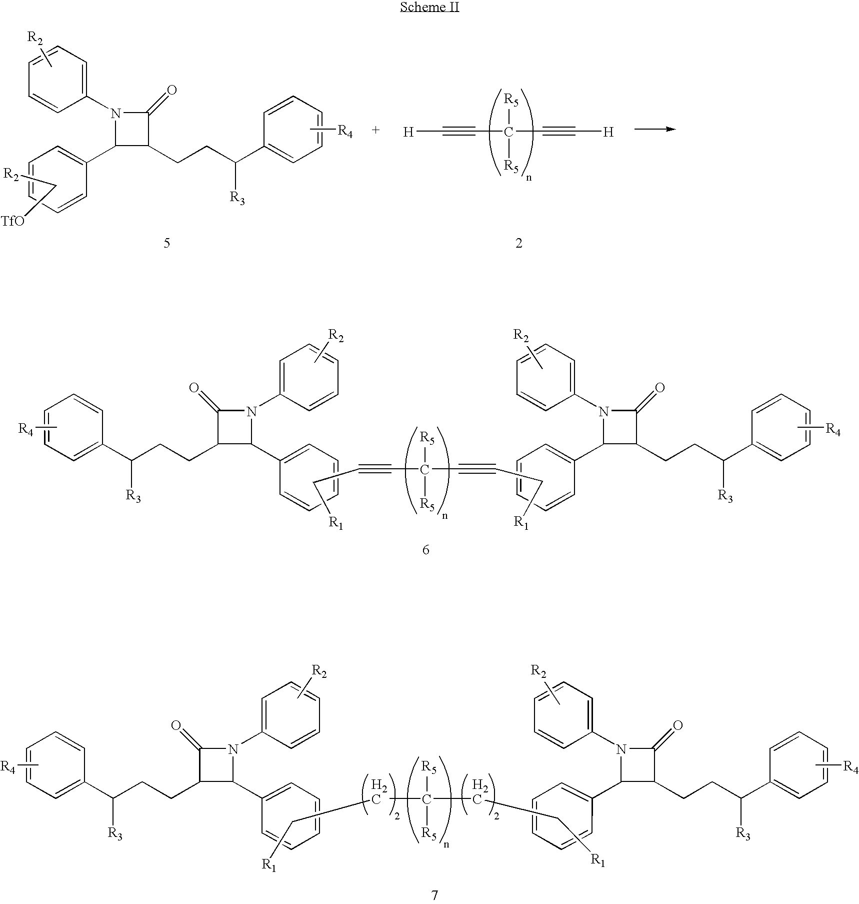

Illustrated in Scheme II is the general method for the preparation of cholesterol absorption inhibitors of the general formula 6 and 7. The procedure commences with the Sonogashira coupling of triflate 5 (derived from the corresponding phenol by treatment with N-phenyltrifluoromethanesulfonimide) with bis-acetylenes of the type 2 to afford the dimeric cholesterol absorption inhibitors of formula 6. Hydrogenation of the triple bonds of 6 in the presence of a suitable catalyst, such as palladium on carbon, affords the saturated cholesterol absorption inhibitors of formula 7.

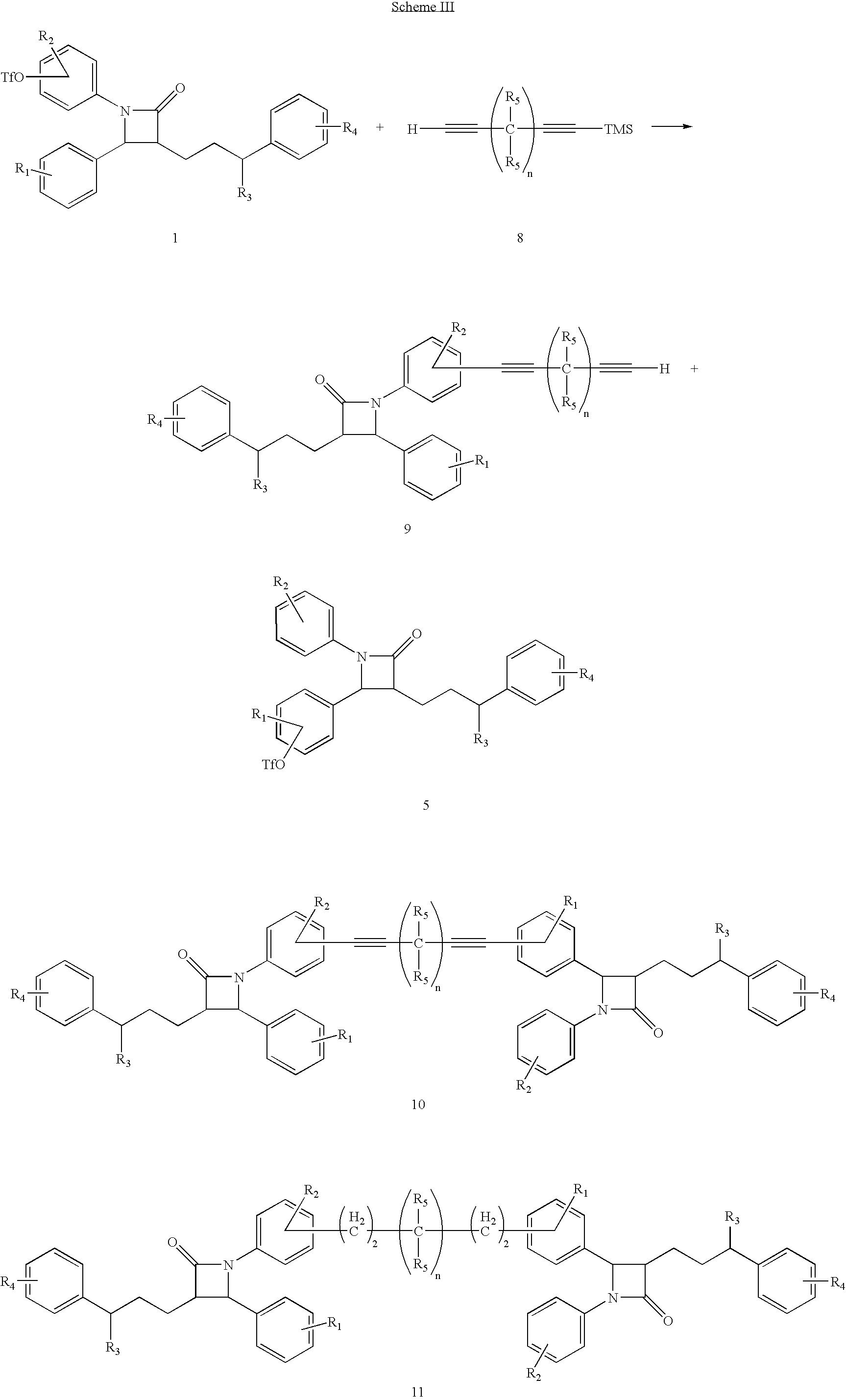

Illustrated in Scheme III is the general method for the preparation of cholesterol absorption inhibitors of the general formula 10 and 11. The procedure commences with the Sonogashira coupling of triflate 1 with the mono-TMS (trimethylsilyl) dehivative of bis-acetylenes 8, (derived from bis-acetylenes of the type 2 by treatment with base followed by chlorotrimethylsilahe), to afford the corresponding coupled product Removal of the trimethylsilyl moiety provides the acetylenes 9. Coupling of 9 with 5 under Sonogashira conditions affords the unsymmetrical cholesterol absorption inhibitors 10. Hydrogenation of the triple bonds of 10 in the presence of a's 'suitable catalyst, such as palladium on carbon, affords the saturated cholesterol absorption inhibitors of formula 11.

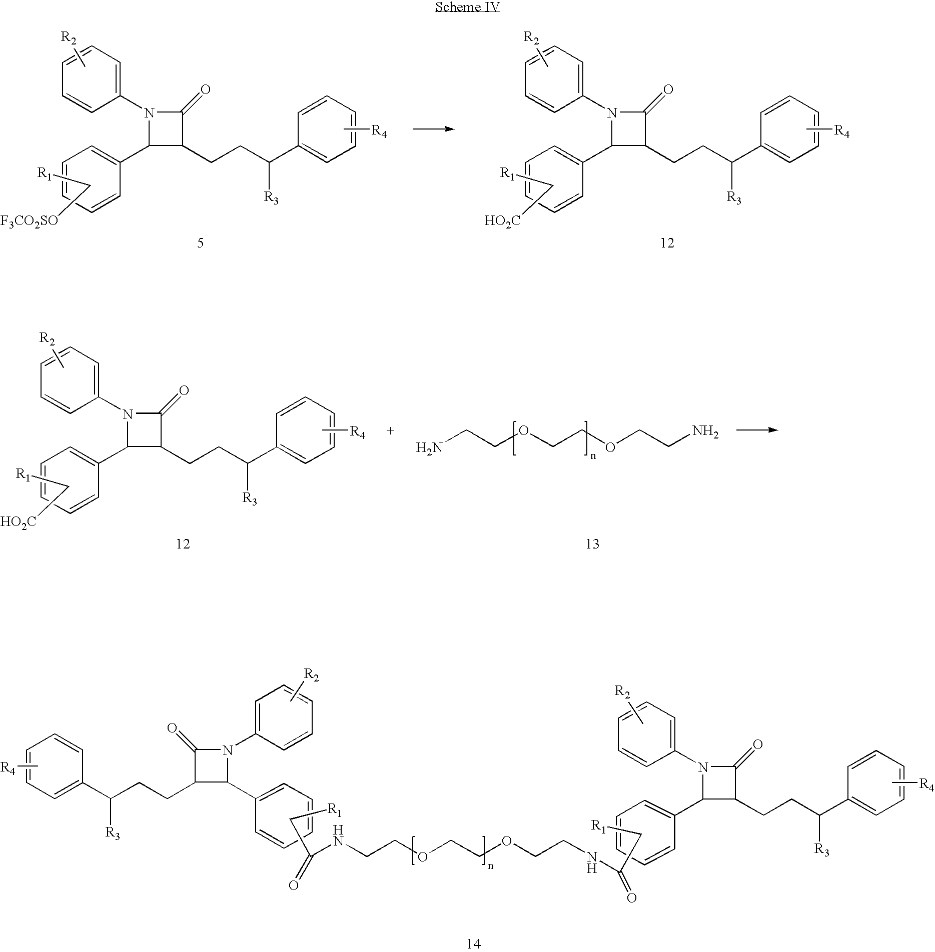

Illustrated in Scheme IV is the general method for the preparation of cholesterol absorption inhibitors of the general formula 14. Triflates 5 are converted into tlieir respective carboxylic acids 12 by dissolving in dimethyl sulfoxide and treatment with carbon monoxide in the presence of palladium II acetate and 1,1-bis-(diphenylphosphino)ferrocene (dppf). Condensation of the acids 12 with diamines such as 13 affords the cholesterol absorption inhibitors of the general structure 14. It is noted that in the above scheme the diamine 13 is for the purposes of illustration and a large variety of aliphatic or aromatic diamines serve as useful coupling partners for the preparation of bis-amide containing cholesterol absorption inhibitors.

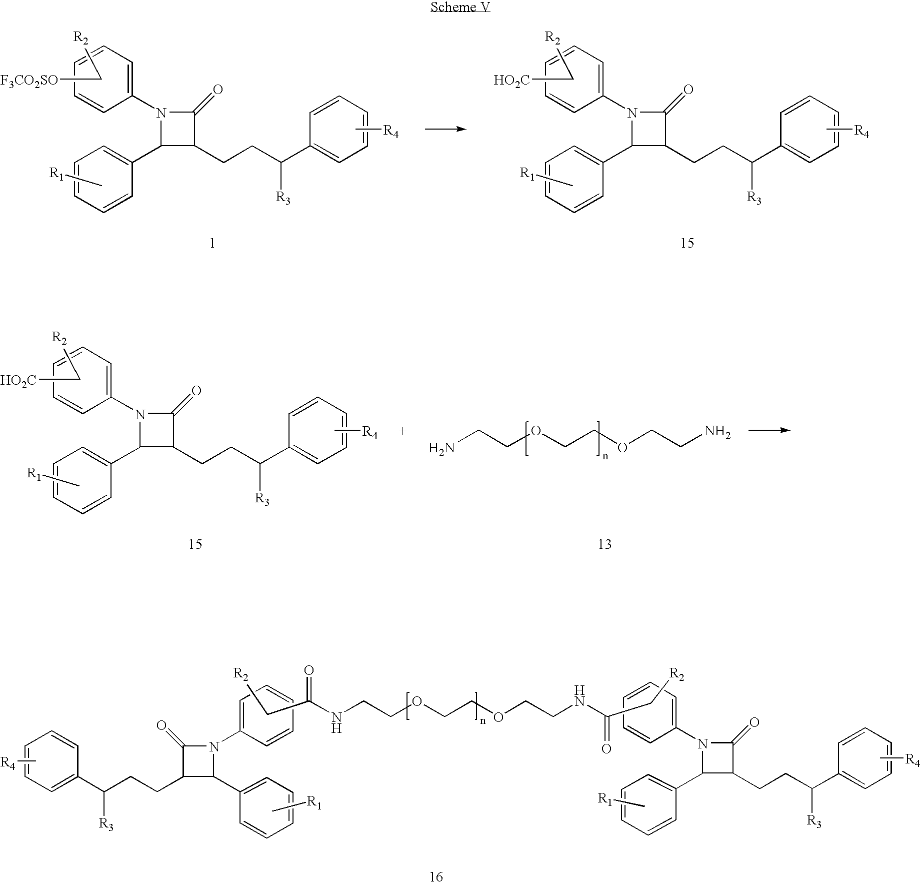

Illustrated in Scheme V is the general method for the preparation of cholesterol absorption inhibitors of the general formula 16. Triflates 1 are converted into their respective carboxylic acids 15 by dissolving in dimethyl sulfoxide and treatment with carbon monoxide in the presence of palladium II acetate and 1,1-bis-(diphenylphosphino)ferrocene (dppf). Condensation of the acids 15 with diamines such as 13 affords the cholesterol absorption inhibitors of the general structure 16. It is noted that in the above scheme the diamine 13 is for the purposes of illustration and a large variety of aliphatic and/or aromatic diamines serve as useful coupling partners for the preparation of bis-amide containing cholesterol absorption inhibitors.

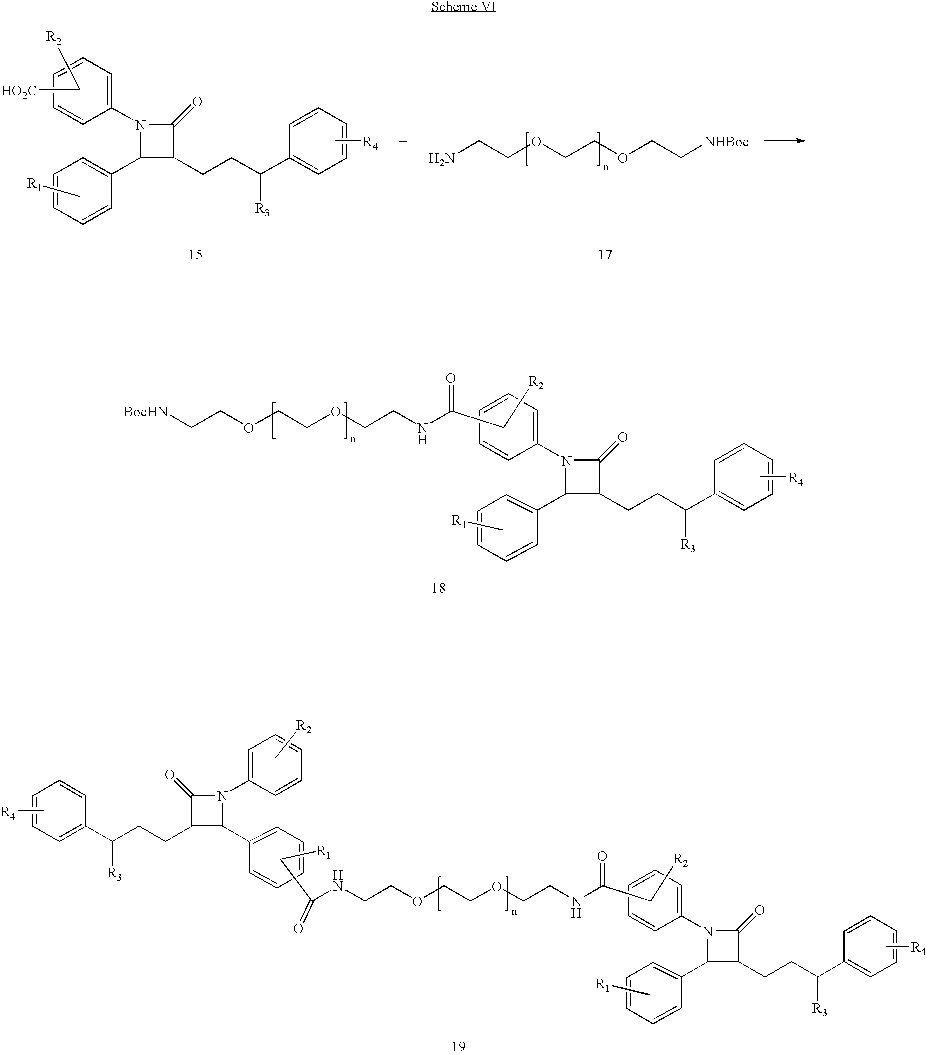

Illustrated in Scheme VI is the general method for the preparation of cholesterol absorption inhibitors of the general formula 19. Condensation of the acids 15 with mono-protected versions of diamines such as 17 affords the cholesterol absorption inhibitors of the general structure 18. To prepare unsymmetrical cholesterol absorption inhibitors of the general formula 19 the protecting group of 18 is removed and the resulting amine coupled with acids of the general formula 12. It is noted that in the above scheme the mono-protected versions of diamines such as 17 is for the purposes of illustration and a large variety of mono-protected aliphatic or aromatic diamines serve as useful coupling partners for the preparation of cholesterol absorption inhibitors.

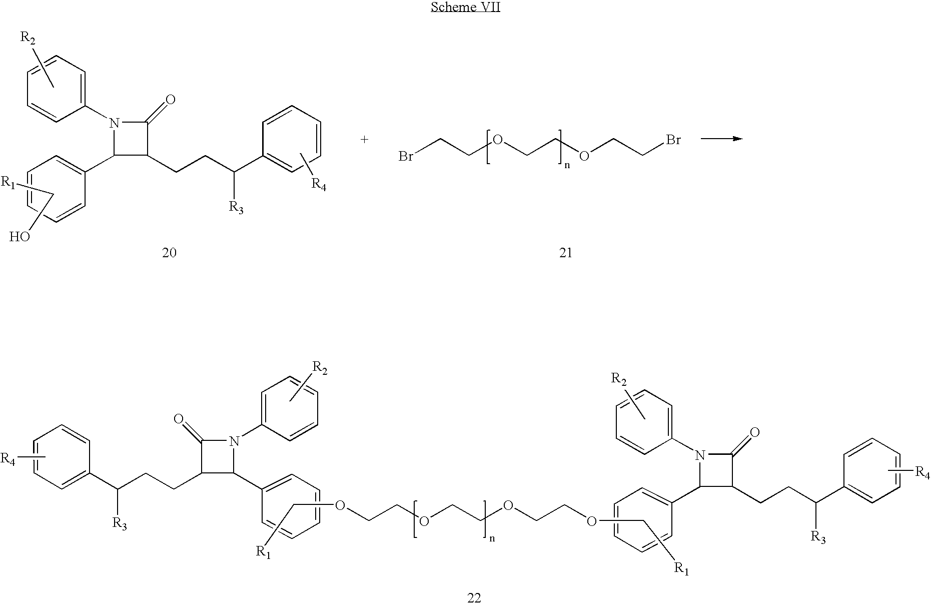

Illustrated in Scheme VII is the general method for the preparation of cholesterol absorption inhibitors of the general formula 22. Condensation of the phenols 20 with dibromides such as 21 affords the cholesterol absorption inhibitors of the general structure 22. It is noted that in the above scheme the dibromo derivatives such as 21 is for the purposes of illustration and a large variety of aliphatic or aromatic dibromo derivatives serve as useful coupling partners for the preparation of cholesterol absorption inhibitors.

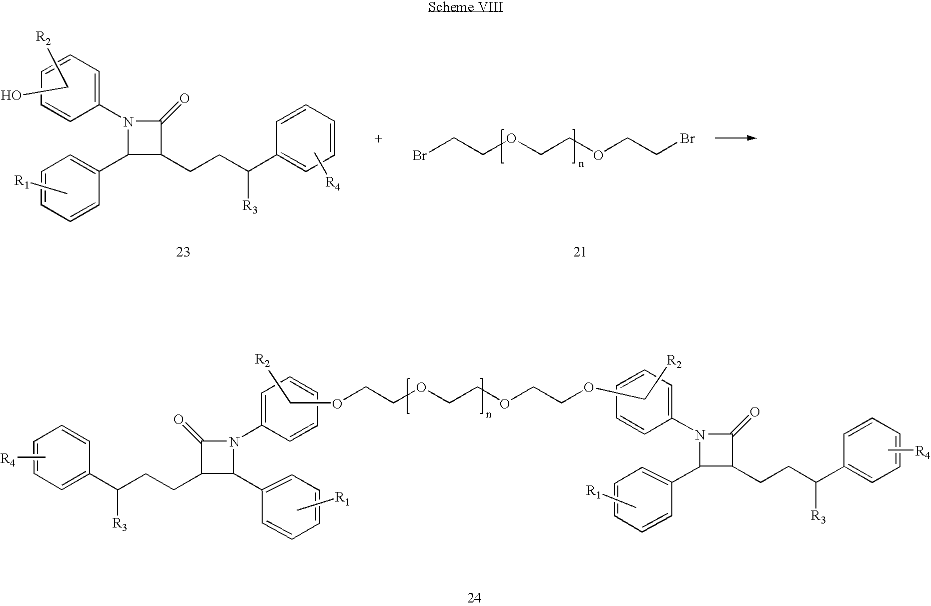

Illustrated in Scheme VIII is the general method for the preparation of cholesterol absorption inhibitors of the general formula 24. Condensation of the phenols 23 with dibromides such as 21 affords the cholesterol absorption inhibitors of the general structure 24. It is noted that in the above scheme the dibromo derivatives such as 21 is for the purposes of illustration and a large variety of aliphatic or aromatic dibromo derivatives serve as useful coupling partners for the preparation of cholesterol absorption inhibitors.

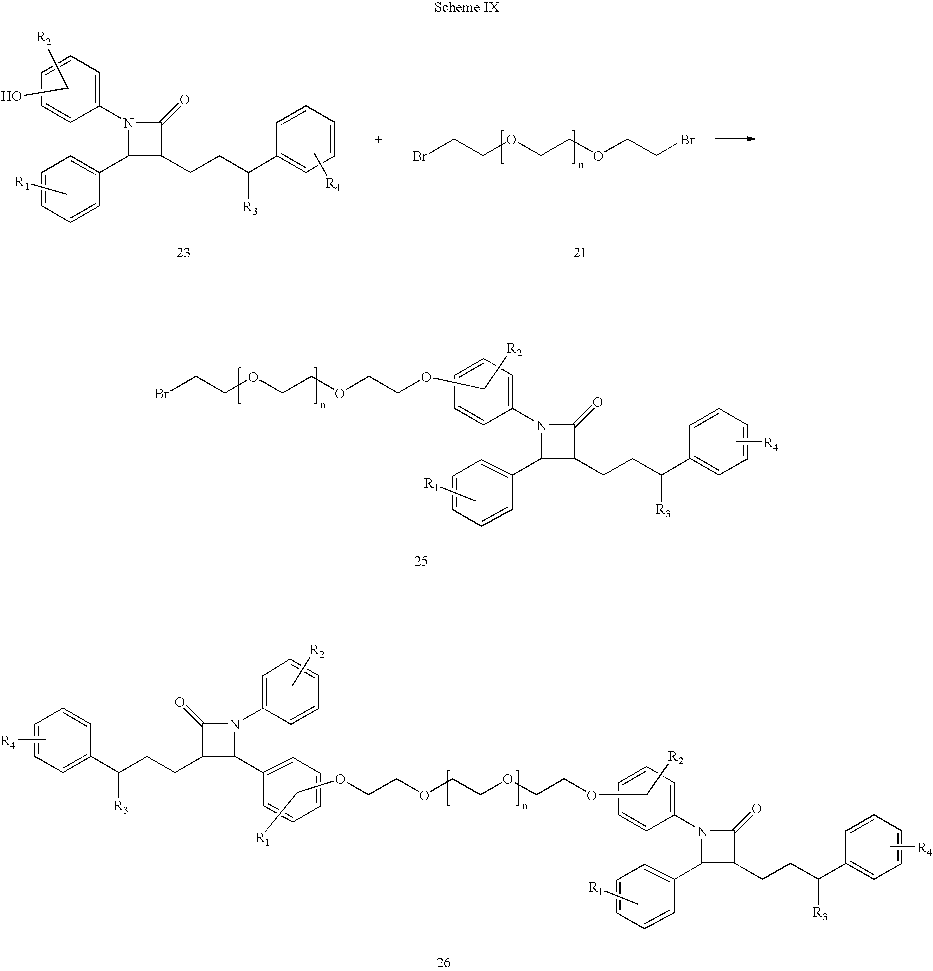

Illustrated in Scheme IX is the general method for the preparation of cholesterol absorption inhibitors of the general formula 26. Condensation of the phenols 23 with dibromides such as 21 affords the bromo derivatives 25. Condensation of 25 with an equivalent of phenol 20 affords the cholesterol absorption inhibitors of the general structure 26. It is noted that in the above scheme the dibromo derivatives such as 21 is for the purposes of illustration and a large variety of aliphatic or aromatic dibromo derivatives serve as useful coupling partners for the preparation of this type of cholesterol absorption inhibitors.

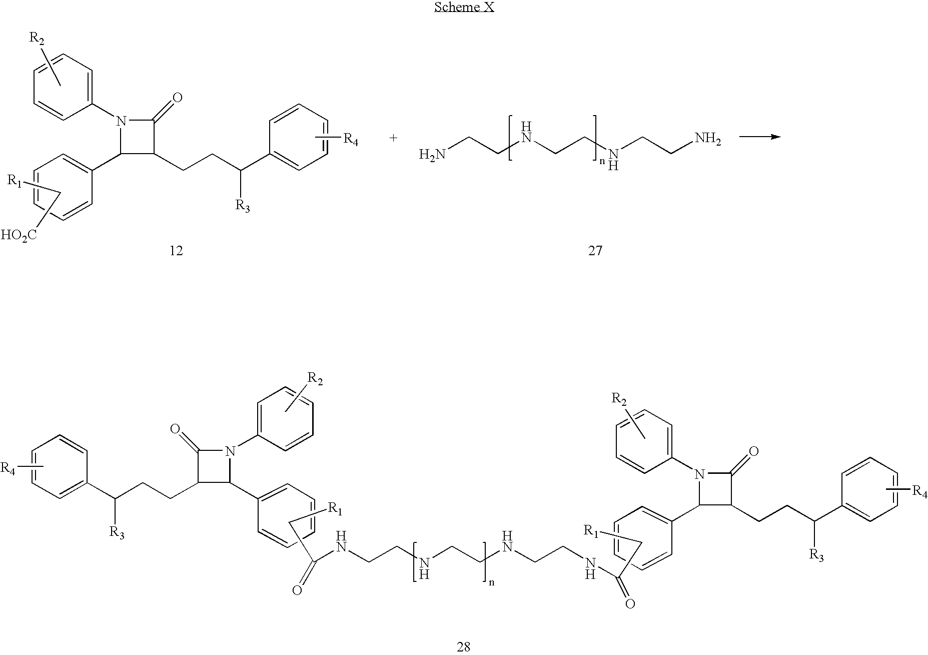

Illustrated in Scheme X is the general method for the preparation of cholesterol absorption inhibitors of the general formula 28. Condensation of the acids 12 with diamines such as 27 affords the cholesterol absorption inhibitors of the general structure 28. It is noted that in the above scheme the diamine 27 is for the purposes of illustration and a large variety of aliphatic and/or aromatic diamines serve as useful coupling partners for the preparation of bis-amide containing cholesterol absorption inhibitors.

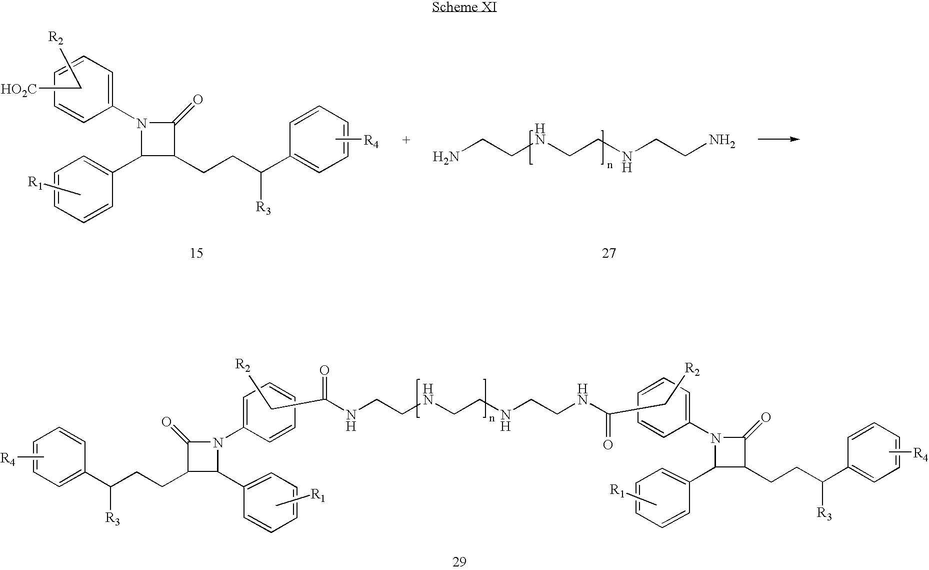

Illustrated in Scheme XI is the general method for the preparation of cholesterol absorption inhibitors of the general formula 29. Condensation of the acids 15 with diamines such as 27 affords the cholesterol absorption inhibitors of the general structure 29. It is noted that in the above scheme the diamine 27 is for the purposes of illustration and a large variety of aliphatic and/or aromatic diamines serve as useful coupling partners for the preparation of bis-amide containing cholesterol absorption inhibitors.

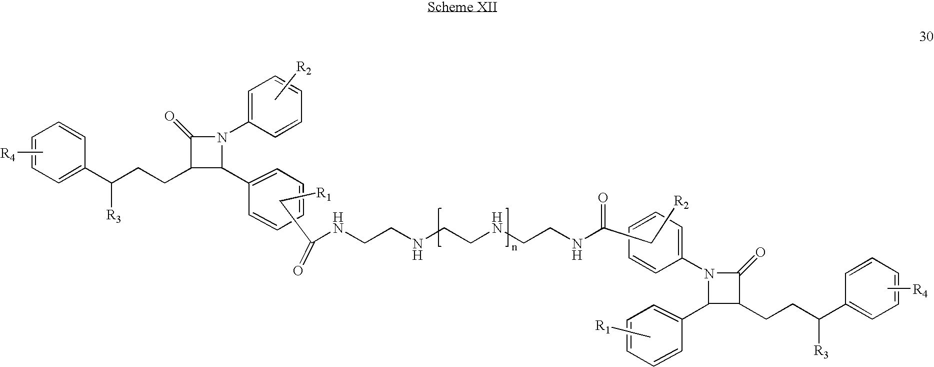

Illustrated in Scheme XII is the general method for the preparation of cholesterol absorption inhibitors of the general formula 30. Condensation of a mixture the acids 12 and 15 with diamines such as 27 affords the cholesterol absorption inhibitors of the general structure 30. It is noted that in the above scheme the diamine 27 is for the purposes of illustration and a large variety of aliphatic and/or aromatic diamines serve as useful coupling partners for the preparation of bis-amide containing cholesterol absorption inhibitors.

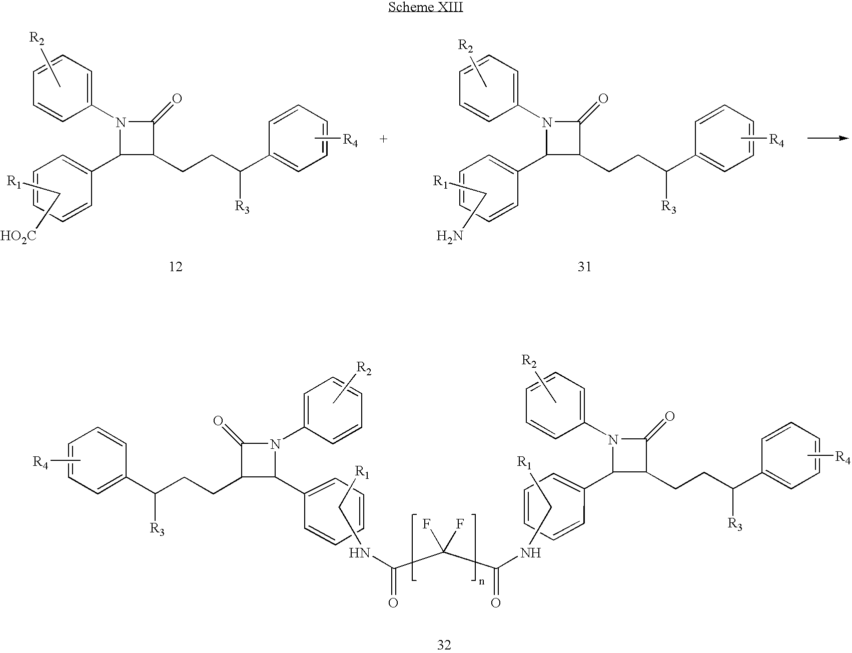

Illustrated in Scheme XIII is the general method for the preparation of cholesterol absorption inhibitors of the general formula 32. Treatmnet of the acids 12 with diphenylphosphoryl azide with warming effects Curtius rearrangement. The incipient isocyanates are then hydrolyzed directly to the anilines 31. Alternatively, the isocyanates can be trapped with an alcohol and the resulting carbamate converted to the corresponding anilines 31. For example, if the isocyanates are trapped with benzyl alcohol the resulting benzyl carbamate can be converted into the anilines 31 by hydrogenolysis. The resulting anilines 31 are then condensed with diacids to provide the cholesterol absorption inhibitors 32. In addition to acids, any activated derivatives of acids such as acid chlorides, activated esters and the like can serve as coupling partners with the anilines. It is noted that in the above scheme the diacids (fluorinated di-acids in the present example) is for the purposes of illustration and a large variety of aliphatic and/or aromatic diacids serve as useful coupling partners for the preparation of bis-amide containing cholesterol absorption inhibitors. In addition, the isocyanates resulting from the Curtius rearrangement of 12 can also be treated with diamines and dialcohols to afford bis-urea and bis-carbamate derived cholesterol absorption inhibitors.

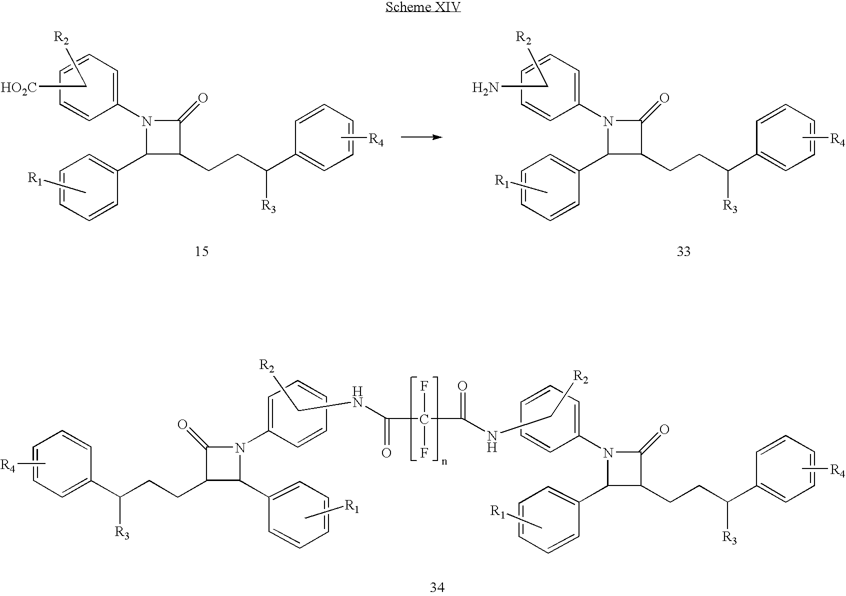

Illustrated in Scheme XIII is the general method for the preparation of cholesterol absorption inhibitors of the general formula 34. Treatment of the acids 15 with diphenylphosphoryl azide with warming effects Curtius rearrangement. The incipient isocyanates are then hydrolyzed directly to the anilines 33. Alternatively, the isocyanates can be trapped with an alcohol and the resulting carbamate converted to the corresponding anilines 33. For example, if the isocyanates are trapped with benzyl alcohol the resulting benzyl carbamate can be converted into the anilines 33 by hydrogenolysis. The resulting anilines 33 are then condensed with diacids to provide the cholesterol absorption inhibitors 34. In addition to acids, any activated derivatives of acids such as acid chlorides, activated esters and the like can serve as coupling partners with the anilines. It is noted that in the above scheme the diacids (fluorinated di-acids in the present example) is for the purposes of illustration and a large variety of aliphatic and/or aromatic diacids serve as useful coupling partners for the preparation of bis-amide containing cholesterol absorption inhibitors. In addition, the isocyanates resulting from the Curtius rearrangement of 15 can also be treated with diamines and dialcohols to afford bis-urea and bis-carbamate derived cholesterol absorption inhibitors.

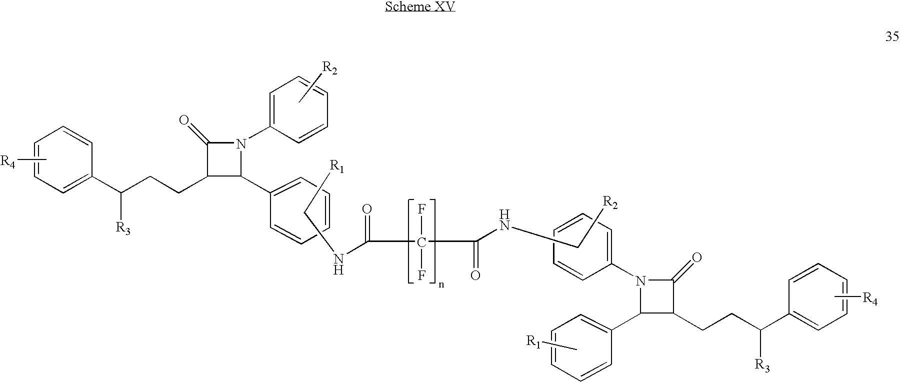

Illustrated in Scheme XV is the structure of cholesterol absorption inhibitors of the general formula 35. Condensation of a mixture of the anilines 31 and 33 with diacids provides the cholesterol absorption inhibitors 35. In addition to acids, any activated derivatives of acids such as acid chlorides, activated esters and the like can serve as coupling partners with the anilines. It is noted that in this procedure the diacids (fluorinated di-acids in the present example) is for the purposes of illustration and a large variety of aliphatic and/or aromatic diacids serve as useful coupling partners for the preparation of bis-amide containing cholesterol absorption inhibitors.

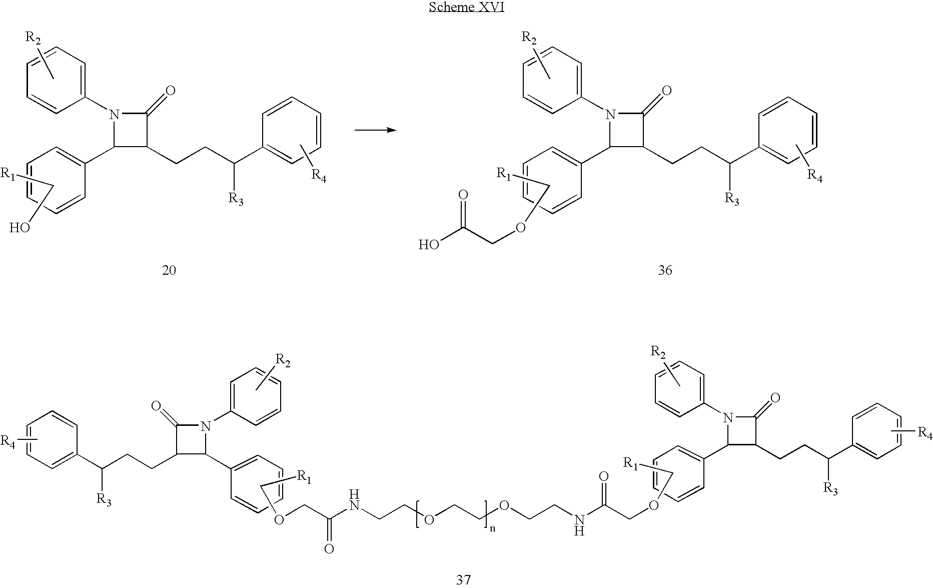

Illustrated in Scheme XVI is the general method for the preparation of cholesterol absorption inhibitors of the general formula 37. Phenols 20 are converted into their respective carboxylic acids 36 by treatment with sodium bromoacetate in the presence of base. The diacids 36 can also be prepared in a two-step process by first condensation of the phenols 20 with tert-butyl bromoacetate and secondly cleaveage of the tert-butyl ester moieties under acidic conditions. Condensation of the acids 36 with diamines such as 13 affords the cholesterol absorption inhibitors of the general structure 37. It is noted that in the above scheme the diamine 13 is for the purposes of illustration and a large variety of aliphatic and/or aromatic diamines serve as useful coupling partners for the preparation of bis-amide containing cholesterol absorption inhibitors.

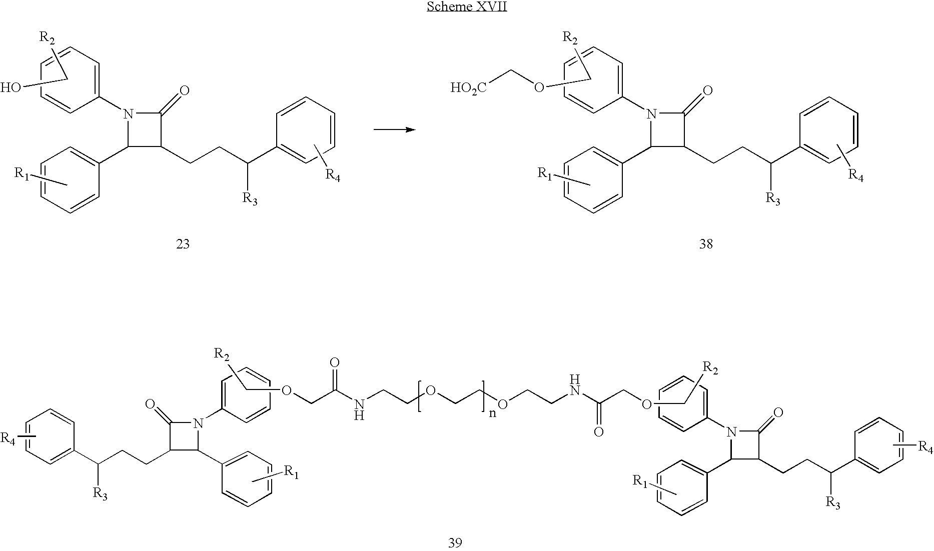

Illustrated in Scheme XVII is the general method for the preparation of cholesterol absorption inhibitors of the general formula 39. Phenols 23 are converted into their respective carboxylic acids 38 by treatment with sodim bromoacetate in the presence of base. The diacids 38 can also be prepared in a two-step process by first condensation of the phenols 23 with tert-butyl bromoacetate and secondly cleaveage of the tert-butyl ester moieties under acidic conditions. Condensation of the acids 38 with diamines such as 13 affords the cholesterol absorption inhibitors of the general structure 39. It is noted that in the above scheme the diamine 13 is for the purposes of illustration and a large variety of aliphatic and/or aromatic diamines serve as useful coupling partners for the preparation of bis-amide containing cholesterol absorption inhibitors.

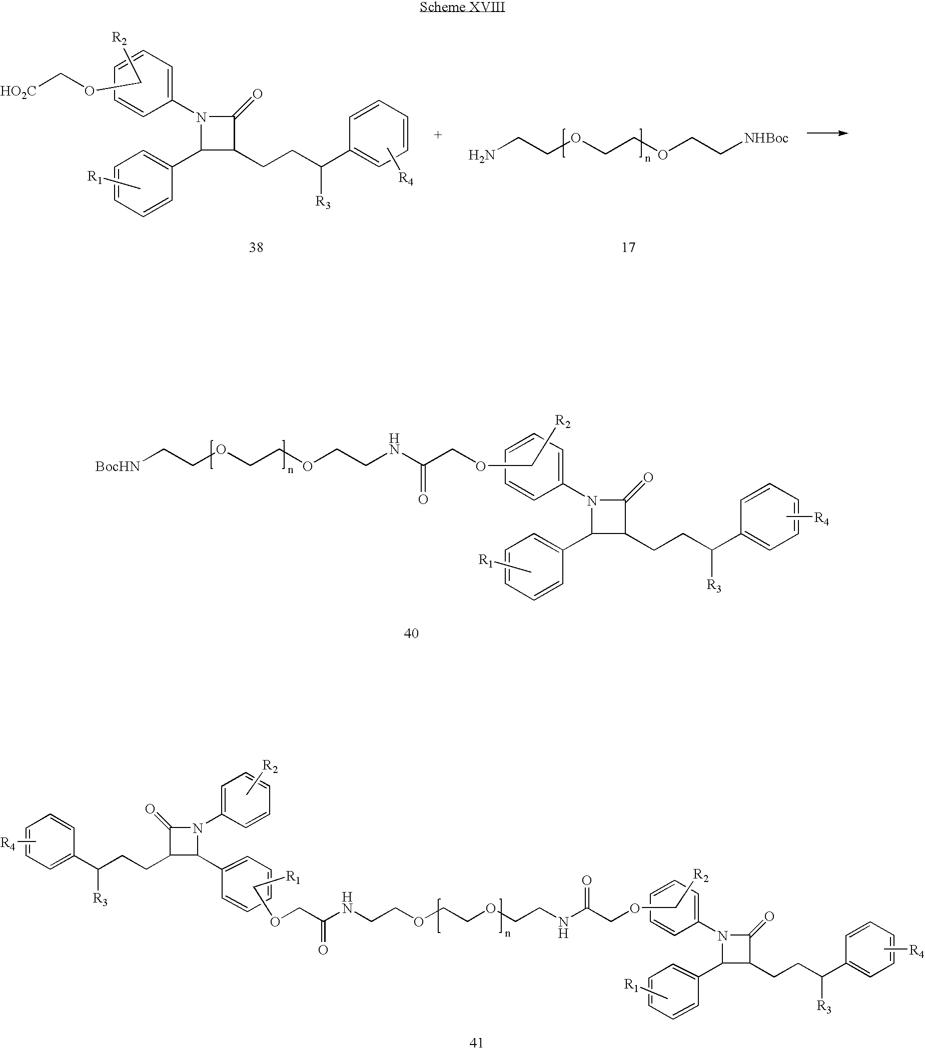

Illustrated in Scheme XVIII is the general method for the preparation of cholesterol absorption inhibitors of the general formula 41. Condensation of the acids 38 with mono-protected versions of diamines such as 17 affords the cholesterol absorption inhibitors of the general structure 40. To prepare unsymmetrical cholesterol absorption inhibitors of the general formula 41 the protecting group of 40 is removed and the resulting amine coupled with acids of the general formula 36. It is noted that in the above scheme the mono-protected versions of diamines such as 17 is for the purposes of illustration and a large variety of mono-protected aliphatic or aromatic diamines serve as useful coupling partners for the preparation of cholesterol absorption inhibitors.

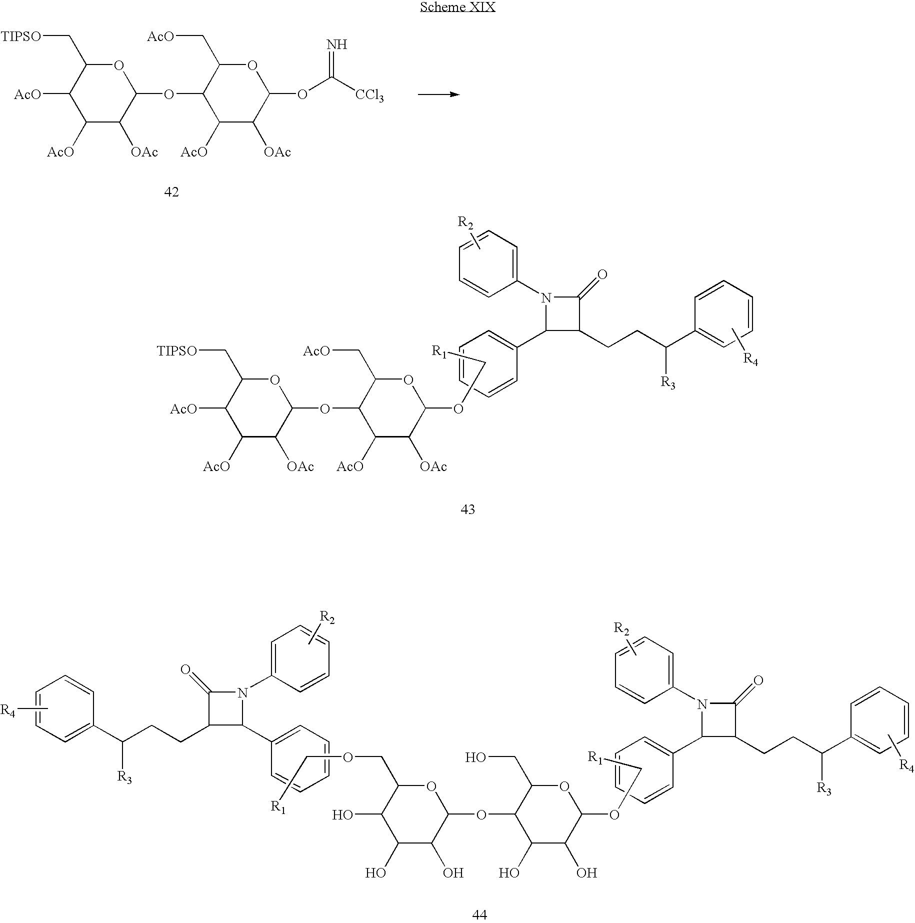

Illustrated in Scheme XIX is the general method for the preparation of cholesterol absorption inhibitors of the general formula 44. The synthetic route commences with the condensation of a protected sugar derivative such as 42 with phenols 20 in the presence of a coupling promoter such as boron trifluoride etherate to afford the carbohydrate derivatives 43. In the present example, the (TIPS, triisopropylsilyl) is selectively deprotected with tetrabutylammonium fluoride to afford the corresponding primary alcohol. The primary alcohol is then converted to the corresponding trifluoromethane sulfonate ester with trifluoromethanesulfonic anhydride and then treated with 20 in the presence of base to afford the dimeric cholesterol absorption inhibitors. Deprotection of the acetate moieties with methanol/triethylamine/water affords the cholesterol absorption inhibitors 44.

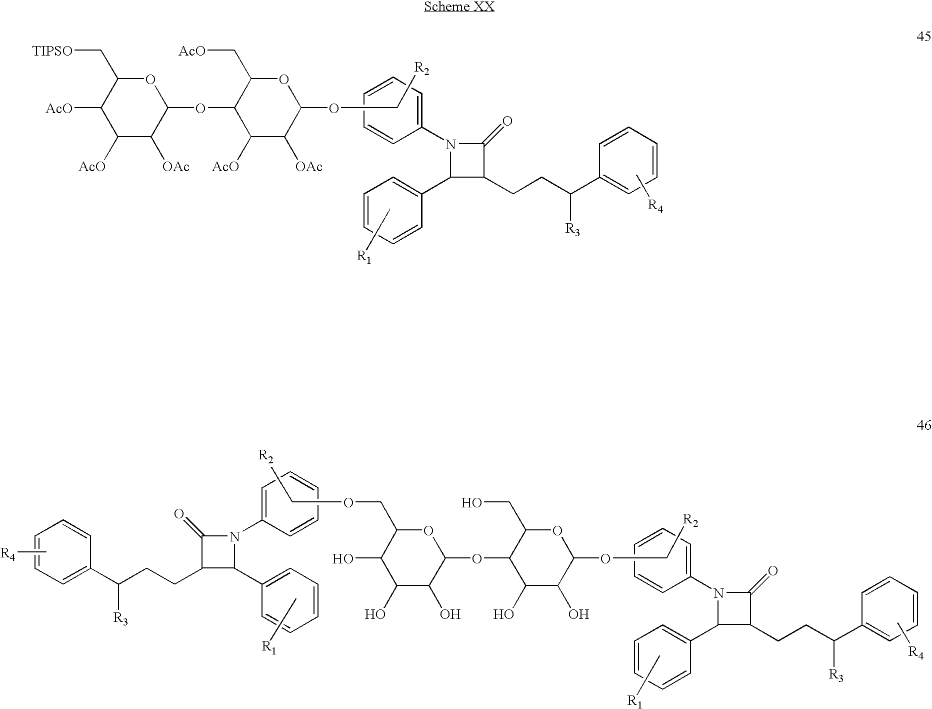

Illustrated in Scheme XX is the general method for the preparation of cholesterol absorption inhibitors of the general formula 46. The synthetic route commences with the condensation of a protected sugar derivative such as 42 with phenols 23 in the presence of a coupling promoter such as boron trifluoride etherate to afford the carbohydrate derivatives 45. In the present example, the (TIPS, triisopropylsilyl) is selectively deprotected with tetrabutylammonium fluoride to afford the corresponding primary alcohol. The primary alcohol is then converted to the corresponding trifluoromethane sulfonate ester with trifluoromethanesulfonic anhydride and then treated with 23 in the presence of base to afford the dimeric cholesterol absorption inhibitors. Deprotection of the acetate moieties with methanol/triethylamine/water affords the cholesterol absorption inhibitors 46.

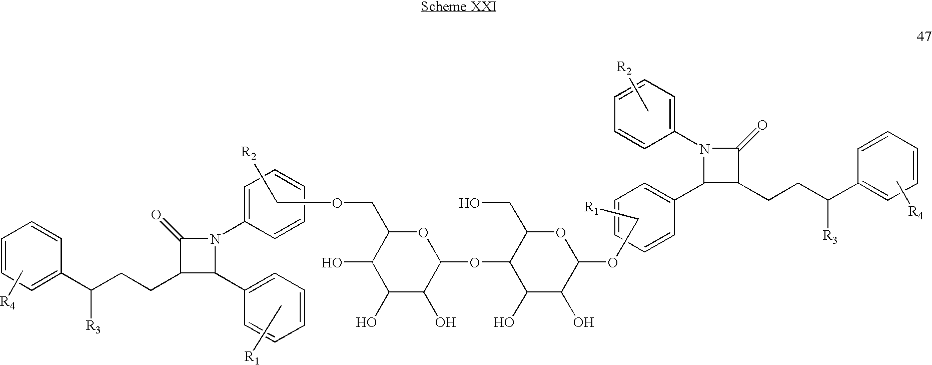

Illustrated in Scheme XXI is the general structure of cholesterol absorption inhibitors of the general formula 47. The synthetic route commences with the protected carbohydrate derivatives 43. The (TIPS, triisopropylsilyl) is selectively deprotected with tetrabutylammonium fluoride to afford the corresponding primary alcohol. The primary alcohol is then converted to the corresponding trifluoromethane sulfonate ester with trifluoromethanesulfonic anhydride and then treated with 23 in the presence of base to afford the dimeric cholesterol absorption inhibitors. Deprotection of the acetate moieties with methanol/triethylamine/water affords the cholesterol absorption inhibitors 47.

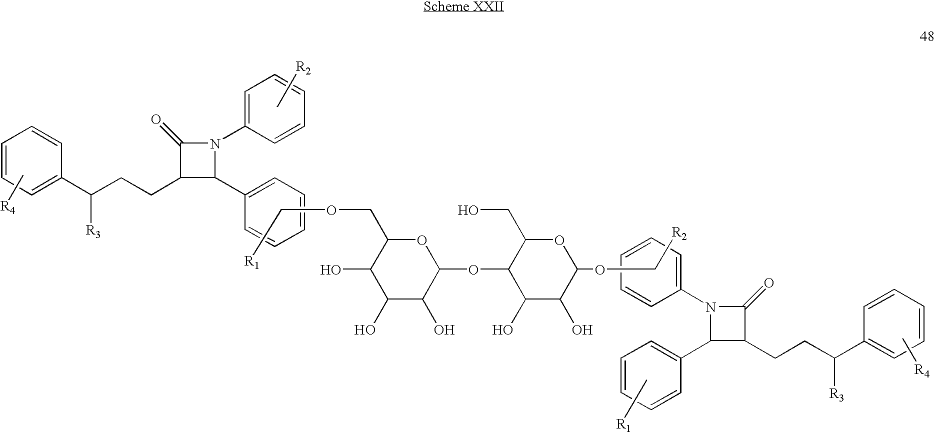

Illustrated in Scheme XXII is the general structure of cholesterol absorption inhibitors of the general formula 48. The synthetic route commences with the protected carbohydrate derivatives 45. The (TIPS, triisopropylsilyl) is selectively deprotected with tetrabutylammoniun fluoride to afford the corresponding primary alcohol. The primary alcohol is then converted to the corresponding trifluoromethane sulfonate ester with trifluoromethanesulfonic anhydride and then treated with 20 in the presence of base to afford the dimeric cholesterol absorption inhibitors. Deprotection of the acetate moieties with methanol/triethylamine/water affords the cholesterol absorption inhibitors 48.

Preparation of 4-{(2S,3R)-1-(4-fluorophenyl)-3-[(3S)-3-(4-fluorophenyl)-3-hydroxypropyl]-4-oxoazetidin-2-yl}phenyl trifluoromethanesulfonate I:

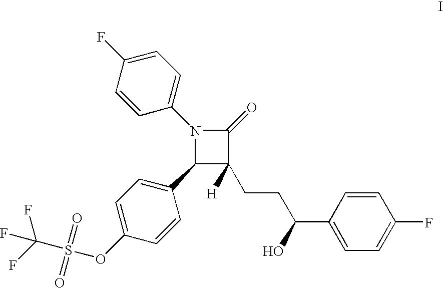

(3R,4S)-1-(4-Fluorophenyl)-3-[(3S)-3-(4-fluorophenyl)-3-hydroxypropyl]-4-(4-hydroxyphenyl)azetidin-2-one (150.4 mg, 0.367 mmol) and 4-dimethylaminopyridine (9.4 mg, 0.077 mmol) were dissolved in methylene chloride (10.0 mL). Triethylamine (100 μL, 72.6 mg, 0.717 mmol) was added via syringe followed by N-phenyltrifluoromethanesulfonimide (143.6 mg, 0.402 imnol) added as a solid. The reaction was stirred for 3.5 h at room temperature and then poured into water (40 mL) and extracted with 1:1 ethyl acetate-hexane (75 mL). The organic layer was washed with water (40 mL) and brine (40 mL), then dried over sodium sulfate, filtered, concentrated and purified by chromatography (12 g silica gel, 10% to 90% ethyl acetate-hexane) to afford 4-{(2S,3R)-1-(4-fluorophenyl)-3-[(3S)-3-(4-fluorophenyl)-3-hydroxypropyl]-4-oxoazetidin-2-yl}phenyl trifluoromethanesulfonate I (190.8 mg, 96% yield) as a clear film (eventually becomes a white solid).

### EXAMPLE 101

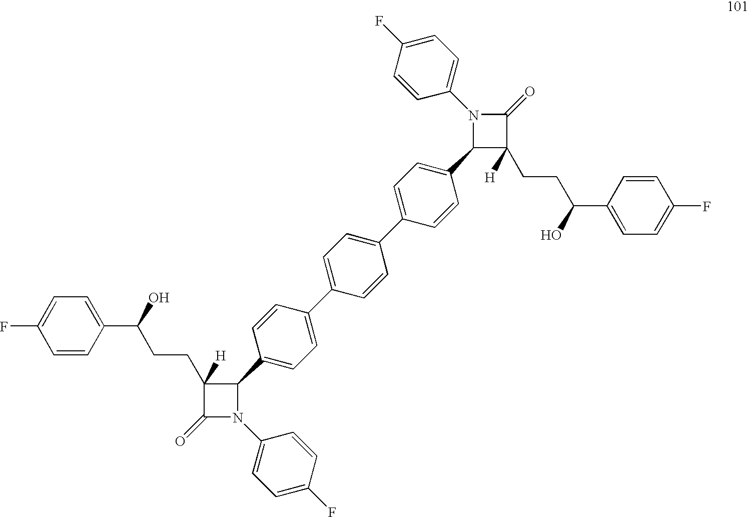

4-{(2S,3R)-1-(4-Fluorophenyl)-3-[(3S)-3-(4-fluorophenyl)-3-hydroxypropyl]-4-oxoazetidin-2-yl}phenyl trifluoromethanesulfonate (100.2 mg, 0.185 mmol) and tetrakis(triphenylphosphine)palladium(0) (10.7 mg, 0.00926 mmol) were dissolved in toluene (1.0 mL). 2.0 M aqueous potassium carbonate (0.185 mL) and a solution of 1,4-benzenediboronic acid (15.2 mg, 0.092 mmol) in ethanol (0.5 mL) were added. The reaction was stirred vigorously for 6 h at refluxing temperature under a nitrogen atmosphere and then diluted with water (2.5 mL), extracted with ethyl acetate (3×10 mL), washed with brine (10 mL), dried over sodium sulfate, filtered, concentrated and purified by chromatography (12 g silica gel, 10% to 100% ethyl acetate-hexane) to afford (3R,4S,3′R,4′S)-4,4′-(1,1′:4′,1″-terphenyl-4,4″-diyl)bis{1-(4-fluorophenyl)-3-[(3S)-3-(4-fluorophenyl)-3-hydroxypropyl]azetidin-2-one} 101 (102 mg, 64% yield) as an off-white solid. NMR ¹H (CDCl₃) 7.7-6.9 (m, 28H), 4.7(m, 2H), 4.6 (s, 2H), 3.2 (m, 2H), 2.1-1.9 (m, 8H).

### EXAMPLE 102

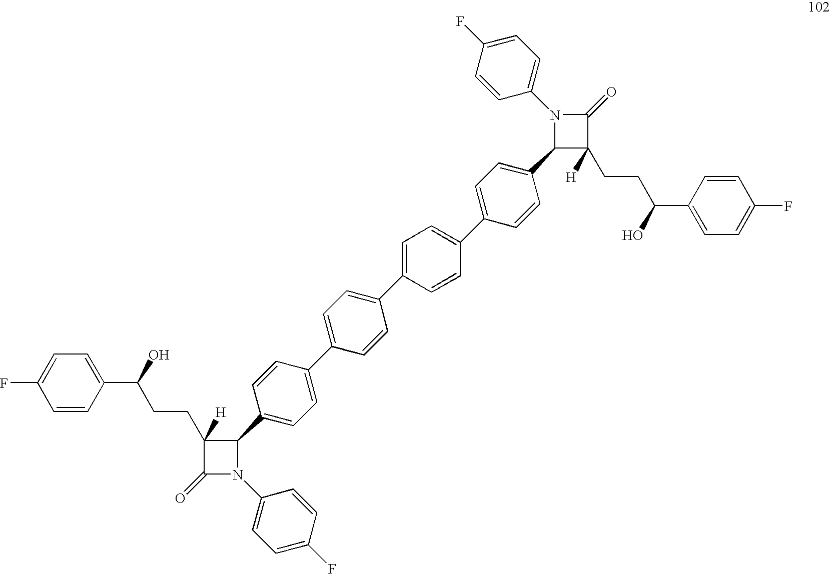

4-{(2S,3R)-1-(4-Fluorophenyl)-3-[(3S)-3-(4-fluorophenyl)-3-hydroxypropyl]-4-oxoazetidin-2-yl}phenyl trifluoromethanesulfonate (80.1 mg, 0.148 mmol) and tetrakis(triphenylphosphine)palladium(0) (8.5 mg, 0.00736 mmol) were dissolved in toluene (1.0 mL). 2.0 M aqueous potassium carbonate (0.148 mL) and a solution of 4,4′-biphenyldiboronic acid (17.9 mg, 0.074 mmol) in ethanol (0.5 mL) were added. The reaction was stirred vigorously for 5 h at refluxing temperature under a nitrogen atmosphere and then diluted with water (2.5 mL), extracted with ethyl acetate (3×10 mL), washed with brine(10 mL), dried over sodium sulfate, filtered, concentrated and purified by chromatography (12 g silica gel, 10% to 100% ethyl acetate-hexane) to afford (3R,4S,3′R,4′S)-4,4′-(1,1′:4′,1″:4″,1′″-quaterphenyl-4,4′″-diyl)bis{1-(4-fluorophenyl)-3-[(3S)-3-(4-fluorophenyl)-3-hydroxypropyl]azetidin-2-one} 102 (58 mg, 42% yield) as an off-white solid. Rf=0.4, silica gel, 1:1 ethyl acetate/hexanes. NMR ¹H (CDCl₃) 7.7-6.9 (m, 32H), 4.7 (m, 2H), 4.6 (s, 2H), 3.2 (m, 2H), 2.1-1.9 (m, 8H).

### EXAMPLE 103

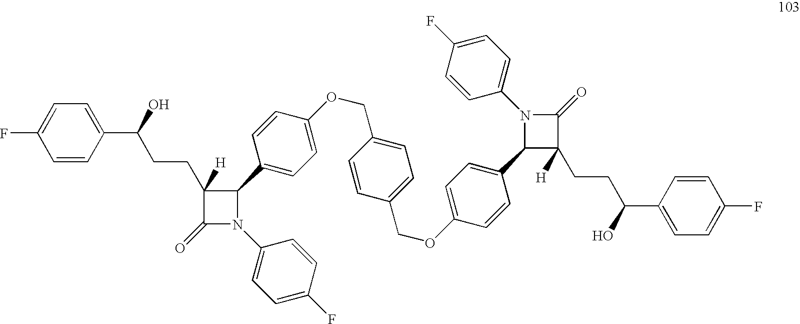

Cesium carbonate (45.7 mg, 0.140 mmol) was lightly flame-dried in a flame-dried flask. When cooled, N,N-dimethylformamide (DMF) (1.4 mL) was added via syringe followed by (3R,4S)-1-(4-fluorophenyl)-3-[(3S)-3-(4-fluorophenyl)-3-hydroxypropyl]-4-(4-hydroxyphenyl)azetidin-2-one (57.6 mg, 0.141 mmol) and finally α,α′-dibromo-para-xylene (31.2 mg, 0.118 mmol) both as solids. The reaction was stirred for 48 h at room temperature, poured into water (40 mL) and extracted with 1:1 ethyl acetate-hexane (40 mL). The organic layer was washed with water (3×25 mL) and brine (25 mL), then dried over sodium sulfate, filtered, concentrated and purified by chromatography (12 g silica gel, 10% to 100% ethyl acetate-hexane) to afford (3R,4S,3′R,4′S)-4,4′-[1,4-phenylenebis(methyleneoxy-4,1-phenylene)]bis{1-(4-fluorophenyl)-3-[(3S)-3-(4-fluorophenyl)-3-hydroxypropyl]azetidin-2-one} 103 (23.6 mg, 22% yield) as an opaque film.

### EXAMPLE 104

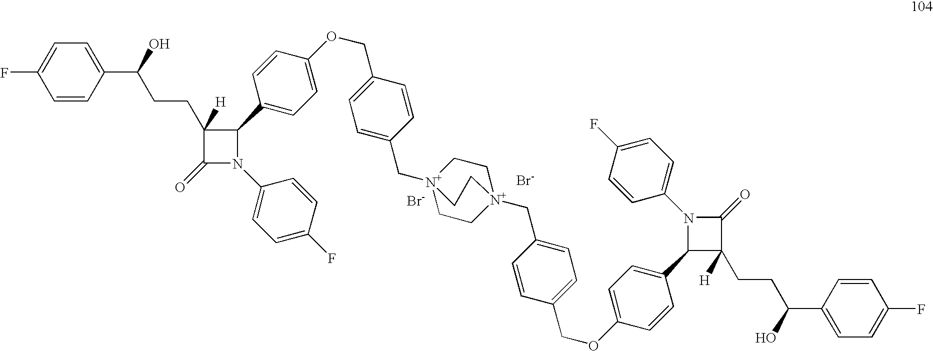

Cesium carbonate (344.7 mg, 1.06 mmol) was lightly flame-dried in a flame-dried flask. When cooled, N,N-dimethylformamide (DMF) (5.0 mL) was added via syringe followed by α,α′-dibromo-p-xylene (826.2 mg, 3.13 mmol) and finally (3R,4S)-1-(4-fluorophenyl)-3-[(3S)-3-(4-fluorophenyl)-3-hydroxypropyl]-4-(4-hydroxyphenyl)azetidin-2-one (254.1 mg, 0.621 mmol) both as solids. The reaction was stirred for 3 h at room temperature, diluted with ethyl acetate (20 mL), filtered through a pad of Celite® and washed with ethyl acetate (100 mL). The solution was transferred to a separatory funnel, washed with water (3×100 mL) and brine (50 mL), dried over sodium sulfate, filtered, concentrated and purified by chromatography (35 g silica gel, 10% to 90% ethyl acetate-hexane) to afford (3R,4S)-4-[4-(4-bromomethylbenzyloxy)phenyl]-1-(4-fluorophenyl)-3-[(3 S)-3 -(4-fluorophenyl)-3-hydroxypropyl]azetidin-2-one 104a (265.3 mg, 72% yield) as a clear film.

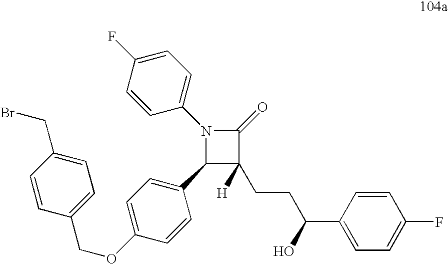

(3R,4S)-4-[4-(4-bromomethyl benzyloxy)phenyl]-1-(4-fluorophenyl)-3-[(3S)-3-(4-fluorophenyl)-3-hydroxypropyl]azetidin-2-one 104a (55.9 mg, 0.094 mmol) was dissolved in dry acetonitrile (2.0 mL). A solution of 1,4-diazabicyclo[2.2.2]octane (9.5 mg, 0.085 mmol) in 0.5 mL of dry acetonitrile was added to the bromide mixture, the reaction was stirred at room temperature for 3 h and then concentrated. The residue was partitioned between water (30 mL) and 1:1 ethyl acetate-hexane (30 mL), shaken to form an emulsion and transferred to two 50-mL Falcon® tubes. The samples were spun at 3000 rpm for 25 min and the aqueous layers are removed carefully via pipette, combined, concentrated at 35° C. and azeotropically dried with methanol (20 mL) to afford 1-{4-[(4-{(2S,3R)-1-(4-fluorophenyl)-3-[(3S)-3-(4-fluorophenyl)-3-hydroxypropyl]-4-oxoazetidin-2-yl}phenoxy)methyl]benzyl}-4-aza-1-azoniabicyclo[2.2.2]octane bromide 104b (54.8 mg, 92% yield) as a clear film.

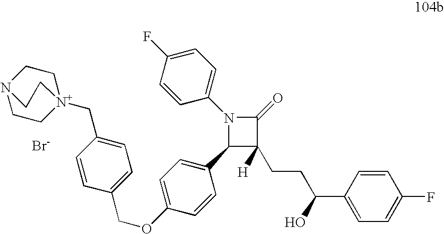

1-{4-[(4-{(2S,3R)-1-(4-fluorophenyl)-3-[(3S)-3-(4-fluorophenyl)-3-hydroxypropyl]-4-oxoazetidin-2-yl}phenoxy)methyl]benzyl}-4-aza-1-azoniabicyclo[2.2.2]octane bromide 104b (79.2 mg, 0.112 mmol) was dissolved in dry acetonitrile (1.0 mL). A solution of (3R,4S)-4-[4-(4-bromomethyl benzyloxy)phenyl]-1-(4-fluorophenyl)-3-[(3S)-3-(4-fluorophenyl)-3-hydroxypropyl]azetidin-2-one 104a (74.2 mg, 0.125 mmol) in 2×3 mL of dry acetonitrile was added to the amine solution, the reaction was stirred at 50° C. for 3 h and then concentrated. The supernatant was decanted off and the remaining residue at the bottom of the flask was triturated with water (20 nL) and ethyl acetate (20 mL) and then azeotropically dried with methanol (20 nL) to afford 1,4-bis{4-[(4-{(2S,3R)-1-(4-fluorophenyl)-3-[(3S)-3-(4-fluorophenyl)-3-hydroxypropyl]-4-oxoazetidin-2-yl}phenoxy)methyl]benzyl}-1,4-diazoniabicyclo[2.2.2]octane dibromide 104 (131.9 mg, 91% yield) as a clear glassy solid.

### EXAMPLE 105

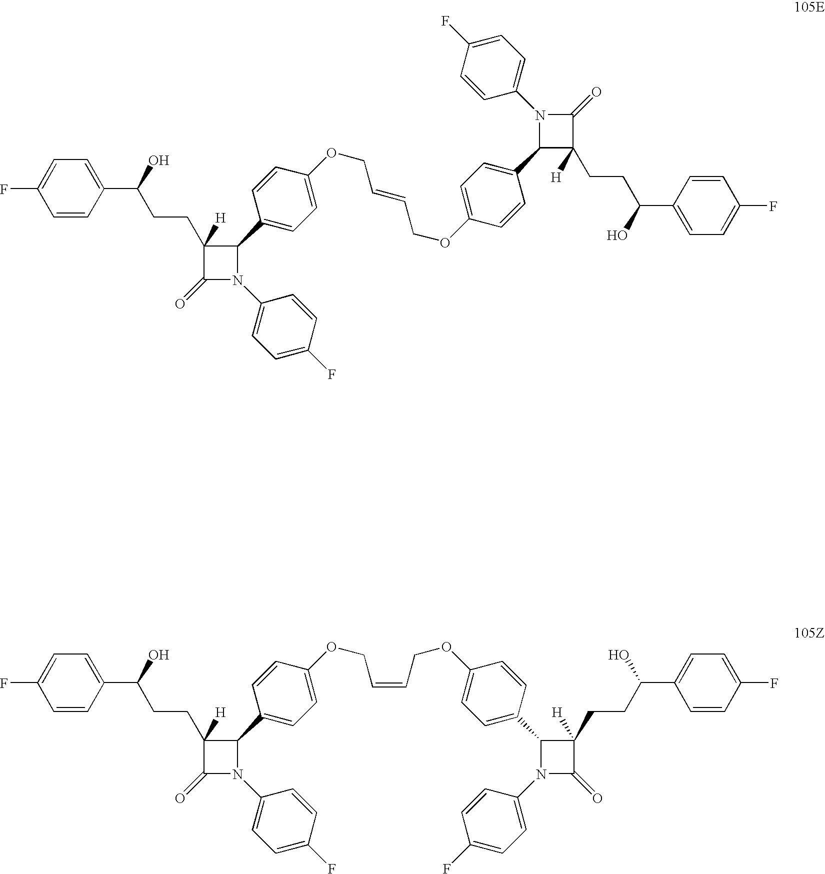

Cesium carbonate (48.8 mg, 0.15 mmol) was lightly flame-dried in a flame-dried flask. When cooled, N,N-dimethylformamide (DMF) (1.25 mL) was added via syringe followed by (3R,4S)-1-(4-fluorophenyl)-3-[(38)-3-(4-fluorophenyl)-3-hydroxypropyl]-4-(4-hydroxyphenyl)azetidin-2-one (51.7 mg, 0.126 mmol) as a solid and finally allyl bromide (25.0 μL, 35.8 mg, 2.96 mmol) via syringe. The reaction was stirred for 12 h at room temperature, diluted with 1:1 ethyl acetate-hexane (50 mL), washed with water (3×50 mL) and brine (50 mL), dried over sodium sulfate, filtered, concentrated and purified by chromatography (12 g silica gel, 10% to 100% ethyl acetate-hexane) to afford (3R,4S)-4-[4-(allyloxy)phenyl]-1-(4-fluorophenyl)-3-[(3S)-3-(4-fluorophenyl)-3-hydroxypropyl]azetidin-2-one 105a (47.4 mg, 84% yield) as a clear film.

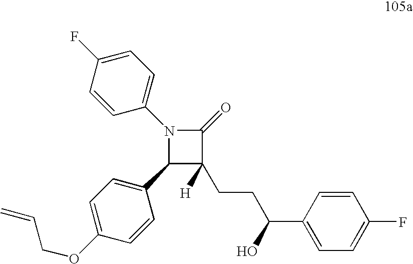

(3R,4S)-4-[4-(Allyloxy)phenyl]-1-(4-fluorophenyl)-3-[(3S)-3-(4-fluorophenyl)-3-hydroxypropyl]azetidin-2-one (47.4 mg, 0.105 mmol) and tricyclohexylphosphine[1,3-bis(2,4,6-trimethylphenyl)-4,5-dihydroimidazol-2-ylidene][benzylidine]ruthenium (IV) dichloride (9.4 mg, 0.011 mmol) were dissolved in methylene chloride (5.0 mL). The reaction was stirred for 38 h at refluxing temperature, cooled, concentrated and purified directly by chromatography (12 g silica gel, 10% to 90% ethyl acetate-hexane) and then by reverse-phase HPLC (21 mm column, 70% to 100% acetonitrile-water) to afford (3R,4S,3′R,4′S)-4,4′-[(2E)-but-2-ene-1,4-diylbis(oxy-4,1-phenylene)]bis{1-(4-fluorophenyl)-3-[(3S)-3-(4-fluorophenyl)-3-hydroxypropyl]azetidin-2-one} 105E and (3R,4S,3′R,4′S)-4,4′-[(2Z)-but-2-ene-1,4-diylbis(oxy-4,1-phenylene)]bis{1-(4-fluorophenyl)-3-[(3S)-3-(4-fluorophenyl)-3-hydroxypropyl]azetidin-2-one} 105Z (20.2 mg, 44% yield) as a while solid (˜1:1 mixture of E/Z olefin isomers).

### EXAMPLE 106

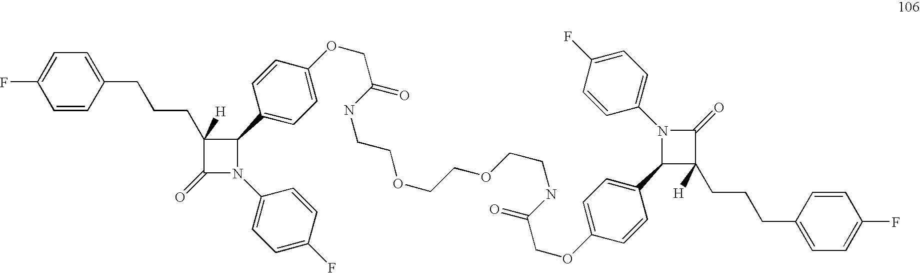

Cesium carbonate (124.6 mg, 0.382 mmol) was lightly flame-dried in a flame-dried flask. When cooled, N,N-dimethylformamide (DMF) (2.5 mL) was added via syringe followed by (3R,4S)-1-(4-fluorophenyl)-3-[(3S)-3-(4-fluorophenyl)-3-hydroxypropyl]-4-(4-hydroxyphenyl)azetidin-2-one (107.9 mg, 0.264 mmol) as a solid and finally tert-butyl bromoacetate (75 μL, 99.1 mg, 0.508 mmol) via syringe. The reaction was stirred for 3.2 h at room temperature, diluted with 1:1 ethyl acetate-hexane (50 mL), washed with water (3×50 mL) and brine (50 mL), dried over sodium sulfate, filtered, concentrated and purified by chromatography (12 g silica gel, 10% to 90% ethyl acetate-hexane) to afford tert-butyl (4-{(2S,3R)-1-(4-fluorophenyl)-3-[(3S)-3-(4-fluorophenyl)-3-hydroxypropyl]-4-oxoazetidin-2-yl}phenoxy)acetate 106a (127.0 mg, 92% yield) as a clear film.

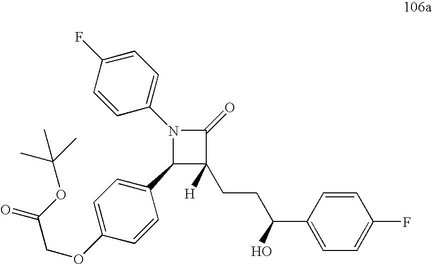

tert-Butyl (4-{(2S,3R)-1-(4-fluorophenyl)-3-[(3S)-3-(4-fluorophenyl)-3-hydroxypropyl]-4-oxoazetidin-2-yl}phenoxy)acetate (127.0 mg, 0.23 mmol) was dissolved in methylene chloride (20 mL). Triethylsilane (2.0 mL, 1.46 g, 12.5 mmol) and trifluoroacetic acid (2.0 mL, 2.96 g, 25.96 mmol) were added via syringe. The reaction was stirred for 72 h at room temperature, diluted with 1:1 ethyl acetate-hexane (50 mL), washed with 2.0 N hydrochloric acid (20 mL), water (50 mL) and brine (50 mL). The solution was dried over sodium sulfate, filtered, concentrated and purified by chromatography (100 g silica gel, 10% methanol-methylene chloride with 0.1% acetic acid) to afford (4-{(2S,3R)-1-(4-fluorophenyl)-3-[3-(4-fluorophenyl)propyl]-4-oxoazetidin-2-yl}phenoxy)acetic acid 106b (89.2 mg, 83% yield) as a clear film.

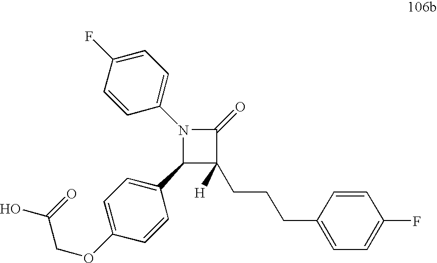

(4-{(2S,3R)-1-(4-fluorophenyl)-3-[3-(4-fluorophenyl)propyl]-4-oxoazetidin-2-yl}phenoxy)acetic acid 106b (89.2 mg, 0.198 mmol) was dissolved in methylene chloride (15 mL). Triethylamine (40 μL, 29.0 mg, 0.287 mmol) and 1-[3-(dimethylamino)propyl]-3-ethylcarbodiimide hydrochloride (54.8 mg, 0.286 mmol) were added to the solution followed by 1,8-diamino-3,6-dioxaoctane (15 μL, 15.2 mg, 0.102 mmol). The reaction was stirred at room temperature for 36 h and then poured into 0.5 N hydrochloric acid (30 mL), extracted with ethyl acetate (30 mL), washed with 0.5 N sodium hydroxide (30 mL), 1.0 M pH 7.4 phosphate buffer (30 mL), brine (30 mL) and dried over sodium sulfate, filtered and concentrated. The residue was purified by chromatography (100 g silica gel, 5% to 10% methanol-methylene chloride) and then by reverse-phase HPLC (21 mm column, 60% to 100% acetonitrile-0.1% trifluoroacetic acid in water) to afford N,N′-[ethane-1,2-diylbis(oxyethane-2,1-diyl)]bis[2-(4-{(2S,3R)-1-(4-fluorophenyl)-3-[3-(4-fluorophenyl)propyl]-4-oxoazetidin-2-yl}phenoxy)acetamide] 106 (3.9 mg, 4% yield) as a clear film.

1. A compound of formula 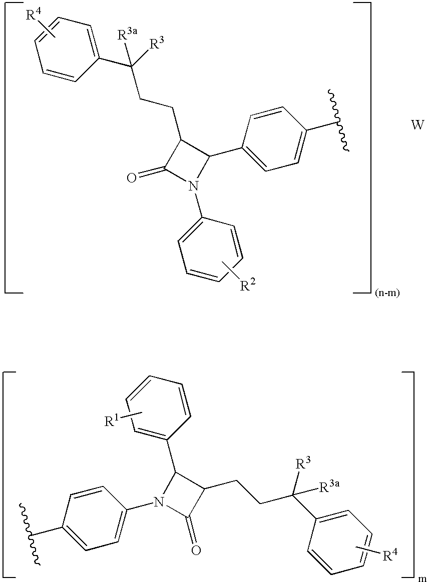
wherein
R¹ and R² are chosen from H, halogen, —OH, loweralkyl, —O-loweralkyl, —CN, —S-loweralkyl, amino, lower alkylamino, alkylsulfonyl, arylsulfonyl, acyl, a sugar, a glucuronide and a sugar carbamate;
R³ is chosen from H, —OH, fluoro and —O-loweralkyl;
R^{3a} is chosen from H and fluoro, or R^{3a} and R³ together are ═O;
R⁴ is chosen from H, halogen, —OH, loweralkyl, —O-loweralkyl, —CN, —S-loweralkyl, amino, lower alkylamino, alkylsulfonyl, arylsulfonyl and acyl; 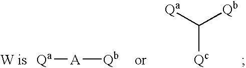
Q^{a}, Q^{b} and Q^{c} are independently chosen from a direct bond, —O—, —S—, —NH—, —CH₂O—, —CH₂NH—, —OCH₂CONH—, —OCH₂COO—, —C(═O)—, —CONH—, —NHCO—, —O(C═O)—, —(C═O)O—, —NHCONH—, —OCONH— and —NHCOO—;
n is 2 or 3;
m is 0, 1, 2 or 3 and m=n; and
A has a valency of n and is chosen from C₂ to C₂₀ hydrocarbon, substituted alkyl of 2 to 20 carbons, perfluoroalkyl of 2 to 20 carbons, substituted aryl, polyaryl of 3 to 20 aryl groups, substituted arylalkyl, oxaalkyl of four to fifty carbons, azaalkyl of four to fifty carbons, thiaalkyl of four to fifty carbons, a residue of an oligopeptide of two to twenty amino acids, a residue of a monosaccharide or of a polysaccharide of 2 to 100 saccharide residues; and, when Q^{a} and Q^{b} are —O(C═O)— or —NHCO—, A may additionally be methylene.

2. A compound according to claim 1 wherein m=zero of formula:

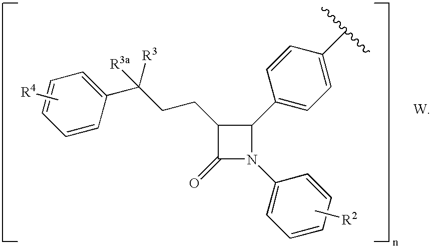

3. A compound according to claim 1 wherein m=n of formula:

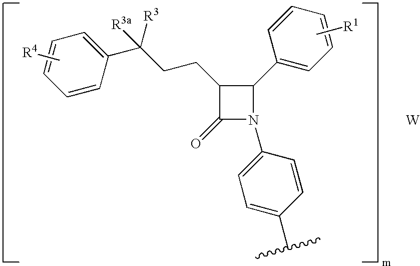

4. A compound according to claim 1 wherein n is 3 and W is trivalent.

5. A compound according to claim 4 wherein Q^{a}, Q^{b} and Q^{c} are independently chosen from —O—, —CH₂O—, —OCH₂CONH—, —OCH₂COO—, —(C═O)O—, and —NHCOO—; and
A is a polysaccharide of 2 to 20 saccharide residues, a branched oxaalkyl of four to fifty carbons or a monoazaalkyl of four to ten carbons.

6. A compound according to claim 4 wherein
Q^{a}, Q^{b} and Q^{c} are independently chosen from —CH₂O—, —CH₂NH—, —OCH₂CONH—, —OCH₂COO—, —CONH—, —NHCO—, —O(C═O)—, —(C═O)O—, —NHCONH—, —OCONH— and —NHCOO—;
and
A is an oligopeptide.

7. A compound according to claim 1 wherein n is 2 and W is divalent of formula:

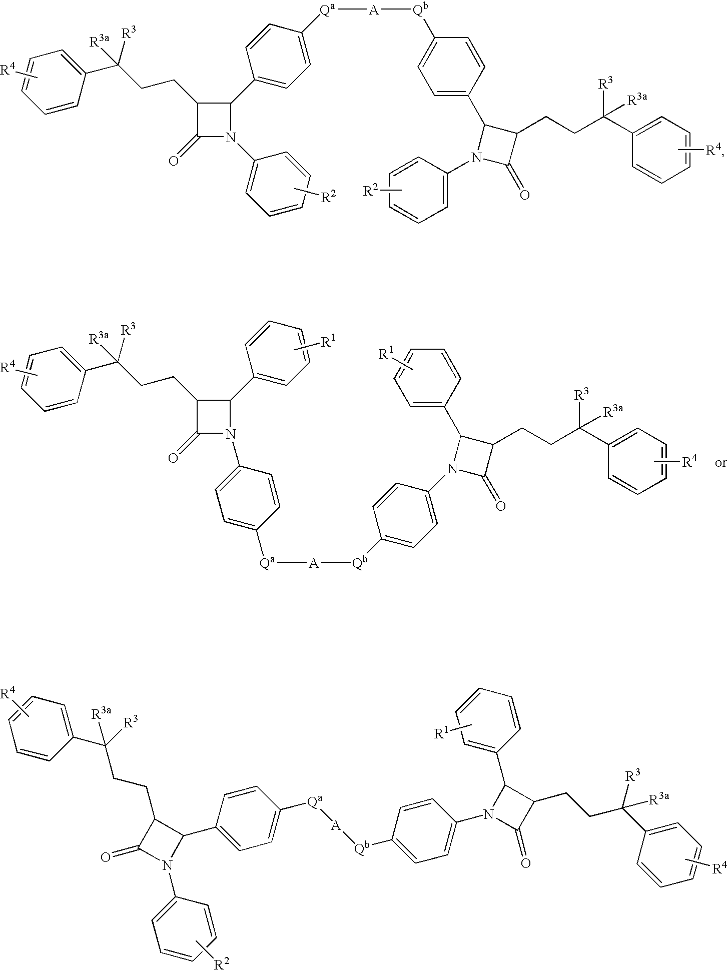

8. A compound according to claim 7 wherein
Q^{a} and Q^{b} are independently chosen from —O—, —CH₂O—, —OCH₂CONH—, —OCH₂COO—, —(C═O)O—, and —NHCOO—; and
A is poly(oxyethylene) or a polysaccharide of 2 to 20 saccharide residues.

9. A compound according to claim 7 wherein
Q^{a} and Q^{b} are independently chosen —CH₂O—, —CH₂NH—, —OCH₂CONH—, —OCH₂COO—, —CONH—, —NHCO—, —O(C═O)—, —(C═O)O—, —NHCONH—, —OCONH— and —NHCOO—;
and
A is an oligopeptide.

10. A compound according to claim 1 wherein
R¹ and R² are chosen from H, halogen, —OH, and methoxy;
R³ is —OH; and
R⁴ is fluoro.

11. A compound according to claim 1 wherein
R¹ and R² are chosen from a sugar, a glucuronide and a sugar carbamate;
R³ is —OH; and
R⁴ is fluoro.

12. A compound according to claim 1 of formula:

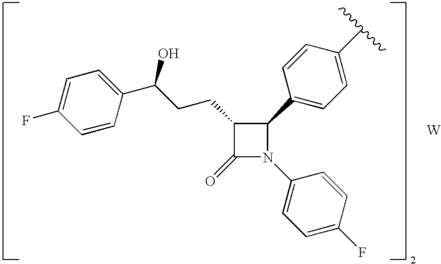

13. A compound according to claim 12 wherein W is chosen from —OCH₂CH═CHCH₂O—,

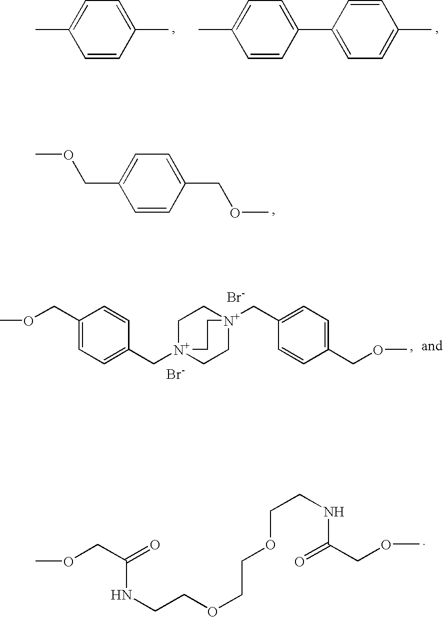

14. A compound according to claim 1 chosen from the group consisting of

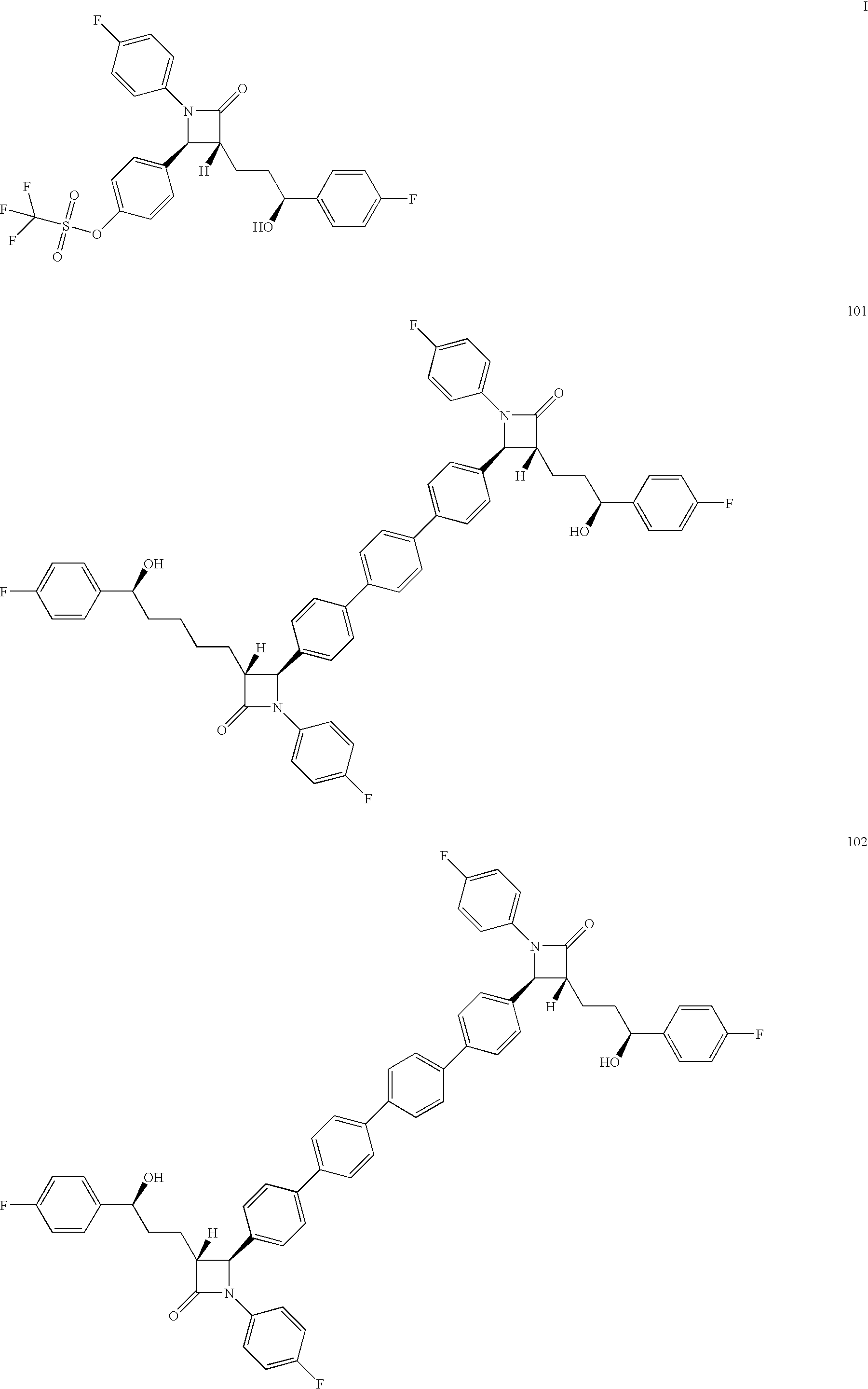

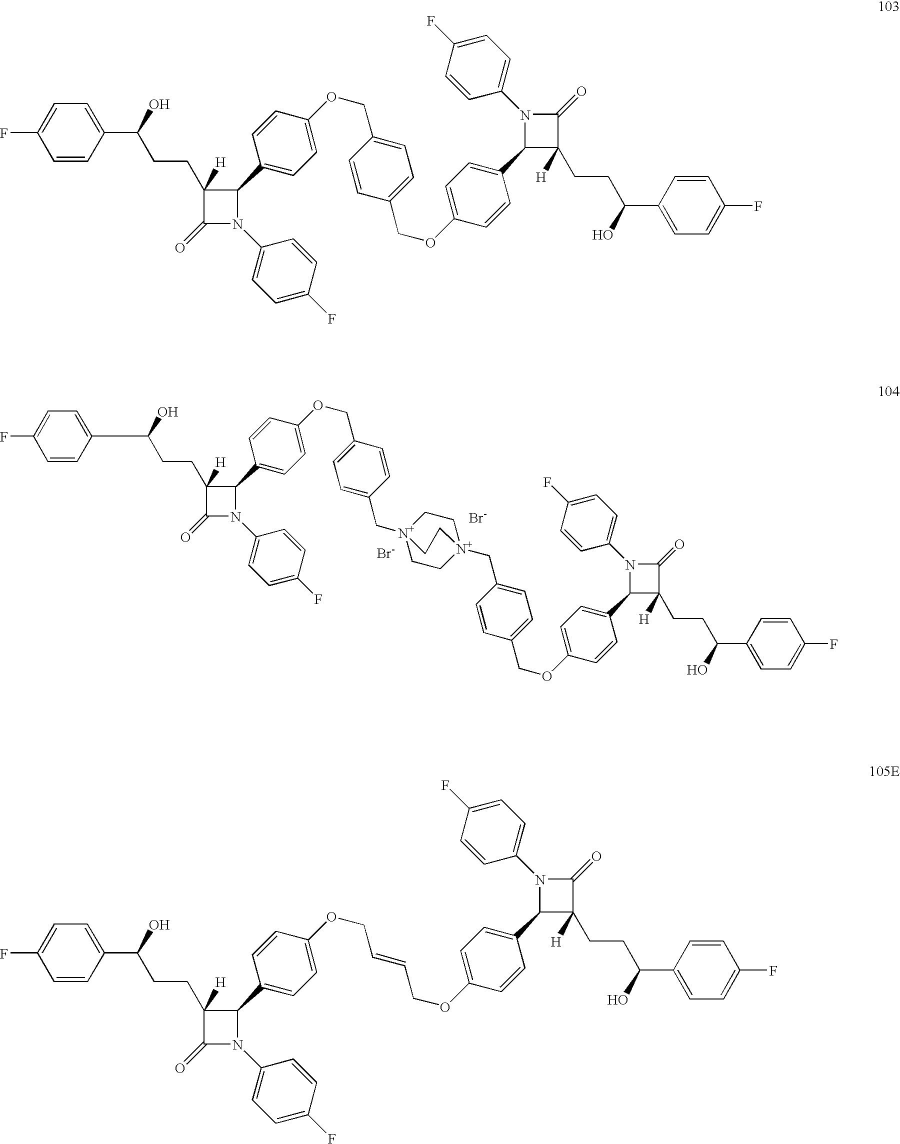

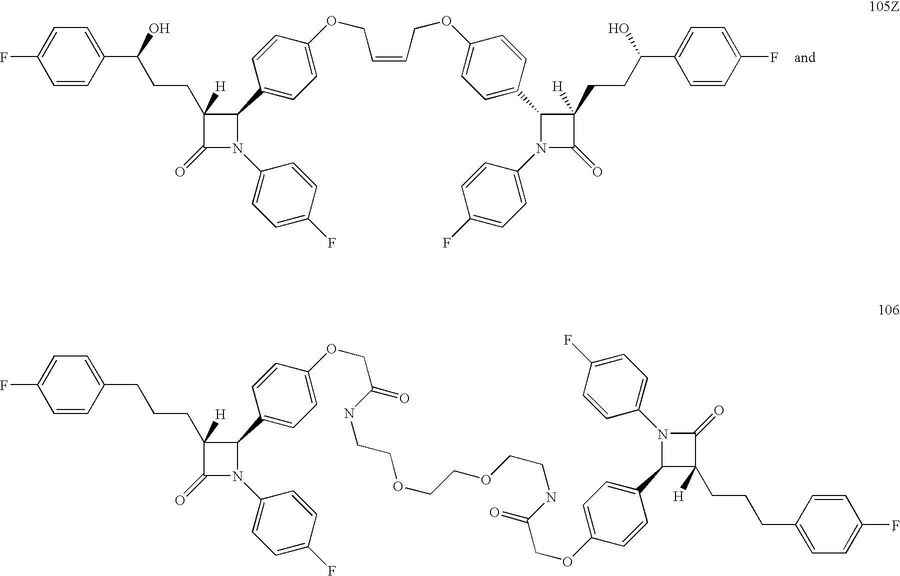

15. A pharmaceutical formulation comprising a compound according to claim 1 and a pharmaceutically acceptable carrier.

16. A pharmaceutical formulation according to claim 15 additionally comprising an inhibitor of cholesterol biosynthesis.

17. A method for treating a disorder of lipid metabolism comprising administering a to a mammal a therapeutically effective amount of a compound according to claim 1.

18. A method according to claim 17, wherein said disorder of lipid metabolism is hyperlipidemia.

19. A method according to claim 17, wherein said disorder of lipid metabolism is arteriosclerosis.

20. A method for inhibiting the absorption of cholesterol from the intestine of a mammal, which comprises administering an effective cholesterol-absorption-inhibiting amount of a compound according to claim 1 to the mammal.

21-30. (canceled)
